(19) United States
(12) Patent Application Publication
(10) Pub. No.: US 2024/0003868 A1
(43) Pub. Date: Jan. 4, 2024

(54) MEASUREMENT DEVICE, MEASUREMENT SYSTEM, INFORMATION PROCESSING METHOD, AND PROGRAM

(71) Applicant: TERUMO KABUSHIKI KAISHA, Tokyo (JP)

(72) Inventor: Satoshi SAWADA, Hadano-shi (JP)

(73) Assignee: TERUMO KABUSHIKI KAISHA, Tokyo (JP)

(21) Appl. No.: 18/467,852

(22) Filed: Sep. 15, 2023

Related U.S. Application Data
(63) Continuation of application No. PCT/JP2022/010497, filed on Mar. 10, 2022.

(30) Foreign Application Priority Data
Mar. 31, 2021 (JP) 2021-061294

(51) Int. Cl. G01N 33/493; A61M 25/10

(52) U.S. Cl. CPC G01N 33/493; A61M 25/10; A61M 2025/0001

(57) ABSTRACT

A measurement device that allows a user to easily change a time axis of a graph indicating parameters measured using a bladder indwelling catheter. The measurement device includes a measurement unit that sequentially measures parameters related to urine on the basis of information obtained by a sensor disposed so as to be contactable with urine to be conducted by a catheter, a display unit that displays the parameters as a graph using a time axis, and a reception unit that receives a change instruction related to the time axis.

### CROSS-REFERENCES TO RELATED APPLICATIONS

This application is a continuation of International Application No. PCT/JP2022/010497 filed on Mar. 10, 2022, which claims priority to Japanese Application No. 2021-061294 filed on Mar. 31, 2021, the entire content of both of which is incorporated herein by reference.

### TECHNOLOGICAL FIELD

The present disclosure generally relates to a measurement device, a measurement system, an information processing method, and a program.

### BACKGROUND DISCUSSION

There is a case where a bladder indwelling catheter is indwelled in a patient who cannot discharge urine by himself, such as immediately after surgery. In addition, a patient in such a condition may develop acute kidney injury.

An oxygen measurement method using a bladder indwelling catheter equipped with an oxygen sensor has been proposed. By measuring an oxygen partial pressure in urine in real time and displaying a time-series graph, or the like, signs of acute kidney injury can be found relatively early (U.S. Patent Application Publication No. 2020-0205718 A).

However, in the oxygen measurement method of U.S. Patent Application Publication No. 2020-0205718 A, a user cannot rather easily change a time axis of an oxygen partial pressure. Thus, the user cannot appropriately confirm long-term trend and short-term trend of an oxygen partial pressure change according to a situation.

### SUMMARY

A measurement device is disclosed that allows a user to relatively easily change a time axis of a graph indicating parameters measured using a bladder indwelling catheter.

A measurement device includes a measurement unit that sequentially measures parameters related to urine on the basis of information obtained by a sensor disposed so as to be contactable with urine to be conducted by a catheter, a display unit that displays the parameters as a graph using a time axis, and a reception unit that receives a change instruction related to the time axis.

In accordance with an aspect, the parameters include at least one of a urine flow rate, a urinary oxygen partial pressure, a carbon dioxide partial pressure, a temperature, a urine potassium concentration, a sodium concentration, a hydrogen ion concentration index, or a creatinine value.

In accordance with another aspect, the parameters include a urinary oxygen partial pressure, and the measurement device comprises a correction unit configured to correct the urinary oxygen partial pressure measured by the measurement unit by at least one of an atmospheric pressure, a pressure in the bladder, a temperature, or chloride concentration in the urine.

In accordance with a further aspect, the measurement unit includes an optical flow rate sensor, an ultrasonic flow rate sensor, a thermal flow rate sensor, or a flow rate measurement unit that measures a urine flow rate on a basis of change in weight of urine that has been conducted.

In accordance with an aspect, the measurement device can further include a second measurement unit configured to measure at least one of urine specific gravity, a pressure in a bladder, a blood flow velocity around a urethra, or a urine flow rate on a basis of information obtained by a sensor disposed at a place not in contact with urine to be conducted by the catheter.

In accordance with another aspect, the measurement device can further include a velocity calculation unit configured to calculate a velocity of urine per body weight, a velocity of urine per body surface area, a mass velocity of oxygen flowing in the catheter per body weight, or a mass velocity of oxygen flowing in the catheter per body surface area.

In accordance with a further aspect, the sensor includes a first sensor disposed in the bladder and a second sensor disposed outside a body.

In accordance with an aspect, the sensor includes two or more oxygen sensors and two or more temperature sensors.

In accordance with another aspect, the sensor includes a plurality of fluorescence sensors each including a phosphor that emits fluorescence corresponding to a predetermined measurement item and includes one fluorescence detection unit that detects fluorescence emitted from the phosphor included in each of the fluorescence sensors.

In accordance with a further aspect, the sensor includes a plurality of fluorescence sensors each including a phosphor that emits fluorescence corresponding to a predetermined measurement item in a case where the phosphor comes into contact with urine, and the sensor includes: a spectroscopic unit that disperses the fluorescence emitted by each of the plurality of fluorescence sensors; and an analysis unit that analyzes each fluorescence dispersed by the spectroscopic unit on a basis of a predetermined algorithm.

In accordance with an aspect, the measurement device further includes an external data acquisition unit configured to acquire data measured using a ventilator, an extracorporeal circulation device, a pulmonary artery catheter, or a left ventricular catheter, and wherein the display unit is configured to display the data acquired by the external data acquisition unit together with the graph indicating the parameters.

In accordance with another aspect, the measurement device further includes a patient information acquisition unit configured to acquire at least one of age, gender, body weight, a serum creatinine value, or a urea nitrogen amount; and a serum creatinine prediction unit configured to predict a future rising level of the serum creatinine value on a basis of the data acquired by the patient information acquisition unit and the data acquired by the external data acquisition unit.

In accordance with a further aspect, wherein in a case where the reception unit receives the change instruction related to the time axis, the external data acquisition unit is configured to acquire data corresponding to a range of the time axis changed on the basis of the change instruction.

In accordance with an aspect, the measurement device further includes an extracorporeal circulation data acquisition unit configured to acquire one or more of a blood storage amount, an infusion amount, a bleeding amount, a blood transfusion amount, a perfusion flow rate, or a perfusion pressure during extracorporeal circulation execution; a circulating blood data acquisition unit configured to acquire one or more of an oxygen partial pressure, a carbon dioxide partial pressure, a potassium concentration, a sodium concentration, a hydrogen ion concentration index, an oxygen supply amount, an oxygen saturation, a hematocrit value, a hemoglobin amount, an oxygen consumption amount, a plasma bicarbonate ion concentration, or a base excess of a circulating blood; and an oxygen supply and demand estimation unit configured to estimate an oxygen supply and demand state of a kidney on a basis of the data acquired by the extracorporeal circulation data acquisition unit, the data acquired by the circulating blood data acquisition unit, and the parameters.

In accordance with another aspect, in a case where the reception unit receives the change instruction related to the time axis, the extracorporeal circulation data acquisition unit and the circulating blood data acquisition unit acquire data corresponding to a range of the time axis changed on a basis of the change instruction.

In accordance with a further aspect, the measurement device further includes a pulmonary artery catheter data acquisition unit configured to acquire one or more of an arterial oxygen content, a cardiac output, or an arterial oxygen saturation measured using a pulmonary artery catheter; and an oxygen supply and demand unit configured to provide an oxygen supply and demand state of the kidney on a basis of the data acquired by the pulmonary artery catheter data acquisition unit and the parameters.

In accordance with an aspect, in a case where the reception unit receives the change instruction related to the time axis, the pulmonary artery catheter data acquisition unit acquires data corresponding to a range of the time axis changed on a basis of the change instruction.

In accordance with another aspect, the measurement device further includes: a drug administration data acquisition unit configured to acquire data from a drug administration device; and a body fluid balance determination unit configured to determine body fluid balance on a basis of the data acquired by the drug administration data acquisition unit and the parameters.

In accordance with a further aspect, the parameters include a urine flow rate or a urinary oxygen partial pressure; the drug administration data acquisition unit includes a diuretic data acquisition unit configured to acquire administration data of a diuretic; and the measurement device comprises a renal function estimation unit configured to estimate a renal function on a basis of change in the urine flow rate associated with administration of a diuretic or change in the urinary oxygen partial pressure associated with administration of a diuretic, the change being determined on a basis of the diuretic administration data acquired by the diuretic data acquisition unit.

In accordance with a further aspect, in a case where the reception unit receives the change instruction related to the time axis, the drug administration data acquisition unit acquires data corresponding to a range of the time axis changed on a basis of the change instruction.

In accordance with an aspect, the measurement device further includes: an inhaled oxygen concentration data acquisition unit configured to acquire inhaled oxygen concentration data; wherein the measurement device calculates a recommended value of the inhaled oxygen concentration on a basis of the inhaled oxygen concentration data acquired by the inhaled oxygen concentration data acquisition unit and the parameters; and wherein in a case where the reception unit receives the change instruction related to the time axis, the inhaled oxygen concentration data acquisition unit acquires data corresponding to a range of the time axis changed on a basis of the change instruction.

In accordance with an aspect, the measurement device further includes: a patient information acquisition unit that acquires at least one of age, gender, weight, a serum creatinine value, or a urea nitrogen amount; and a renal function deterioration risk prediction unit that predicts a renal function deterioration risk within seven days after surgery or within 90 days after surgery on a basis of the data acquired by the patient information acquisition unit and the parameters.

In accordance with another aspect, the display unit displays a therapeutic guideline based on the parameters together with the graph indicating the parameters.

In accordance with another aspect, the measurement device further includes: a sensor eigenvalue acquisition unit that acquires a sensor eigenvalue defined uniquely for the sensor; and a calibration unit that calibrates the sensor on a basis of the sensor eigenvalue.

In accordance with a further aspect, a measurement system comprising a sensor and a measurement device, the sensor being disposed so as to be contactable with urine to be conducted by a catheter, and the measurement device comprising: a sensor data acquisition unit configured to acquire sensor data from the sensor; a measurement unit configured to sequentially measure parameters related to urine on a basis of the sensor data acquired by the sensor data acquisition unit; a display unit configured to display the parameters as a graph using a time axis; and a reception unit configured to receive a change instruction related to the time axis.

In accordance with an aspect, an information processing method comprising acquiring information from a sensor contactable with urine to be conducted by a catheter; sequentially measuring parameters related to urine using the acquired information; and displaying the parameters as a graph using a time axis; and receiving a change instruction related to the time axis.

In accordance with another aspect, a non-transitory computer-readable medium storing a computer program executed by a computer processor to execute a process comprising: acquiring information from a sensor contactable with urine to be conducted by a catheter; sequentially measuring parameters related to urine using the acquired information; and displaying the parameters as a graph using a time axis; and receiving a change instruction related to the time axis.

In one aspect, it is possible to provide a measurement device that allows a user to relatively easily change a time axis of a graph indicating parameters measured using a bladder indwelling catheter.

### BRIEF DESCRIPTION OF THE DRAWINGS

FIG. 1 is an explanatory view for explaining a configuration of a measurement system.

FIG. 2 is an explanatory view illustrating the configuration of the measurement system.

FIG. 3 is an explanatory view for explaining a configuration of fluorescence measurement equipment.

FIG. 4 is an explanatory view for explaining record layout of a measurement value database (DB).

FIG. 5 is a flowchart for explaining flow of processing of a program.

FIG. 6 is a flowchart for explaining flow of the processing of the program.

FIG. 7 is an explanatory view illustrating an example of a display screen.

FIG. 8 is an explanatory view illustrating an example of the display screen.

FIG. 9 is an explanatory view illustrating an example of the display screen.

FIG. 10 is an explanatory view illustrating an example of the display screen.

FIG. 11 is an explanatory view illustrating an example of the display screen.

FIG. 12 is an explanatory view illustrating an example of the display screen.

FIG. 13 is an explanatory view illustrating a modification of the display screen.

FIG. 14 is an explanatory view illustrating a modification of the display screen.

FIG. 15 is an explanatory view illustrating a modification of the display screen.

FIG. 16 is an explanatory view for explaining a modification of a kidney icon.

FIG. 17 is an explanatory view illustrating a modification of the display screen.

FIG. 18 is an explanatory view for explaining a modification of the fluorescence measurement equipment.

FIG. 19 is a flowchart for explaining flow of processing of a program according to a modification.

FIG. 20 is an explanatory view for explaining a configuration of fluorescence measurement equipment of a second embodiment.

FIG. 21 is an explanatory view for explaining a method of detecting abnormality of a measurement result.

FIG. 22 is an explanatory view for explaining the method of detecting abnormality of the measurement result.

FIG. 23 is a flowchart for explaining flow of processing of a program of the second embodiment.

FIG. 24 is an explanatory view for explaining record layout of an index database (DB).

FIG. 25 is a flowchart for explaining flow of processing of a program of a third embodiment.

FIG. 26 is an explanatory view illustrating an example of a display screen of the third embodiment.

FIG. 27 is an explanatory view illustrating an example of the display screen of the third embodiment.

FIG. 28 is an explanatory view illustrating an example of the display screen of the third embodiment.

FIG. 29 is an explanatory view illustrating an example of a display screen of a fourth embodiment.

FIG. 30 is an explanatory view illustrating an example of the display screen of the fourth embodiment.

FIG. 31 is an explanatory view illustrating an example of the display screen of the fourth embodiment.

FIG. 32 is an explanatory view illustrating an example of the display screen of the fourth embodiment.

FIG. 33 is an explanatory view illustrating an example of the display screen of the fourth embodiment.

FIG. 34 is an explanatory view illustrating an example of the display screen of the fourth embodiment.

FIG. 35 is an explanatory view illustrating an example of the display screen of the fourth embodiment.

FIG. 36 is an explanatory view for explaining a configuration of fluorescence measurement equipment of a fifth embodiment.

FIG. 37 is an explanatory view for explaining a configuration of a measurement device of a sixth embodiment.

FIG. 38 is a functional block diagram of a measurement system of a seventh embodiment.

### DETAILED DESCRIPTION

Set forth below with reference to the accompanying drawings is a detailed description of embodiments of a measurement device, a measurement system, an information processing method, and a program. In the drawings, similar components are denoted by the same reference signs, and the detailed description of the similar components will be appropriately omitted.

### First Embodiment

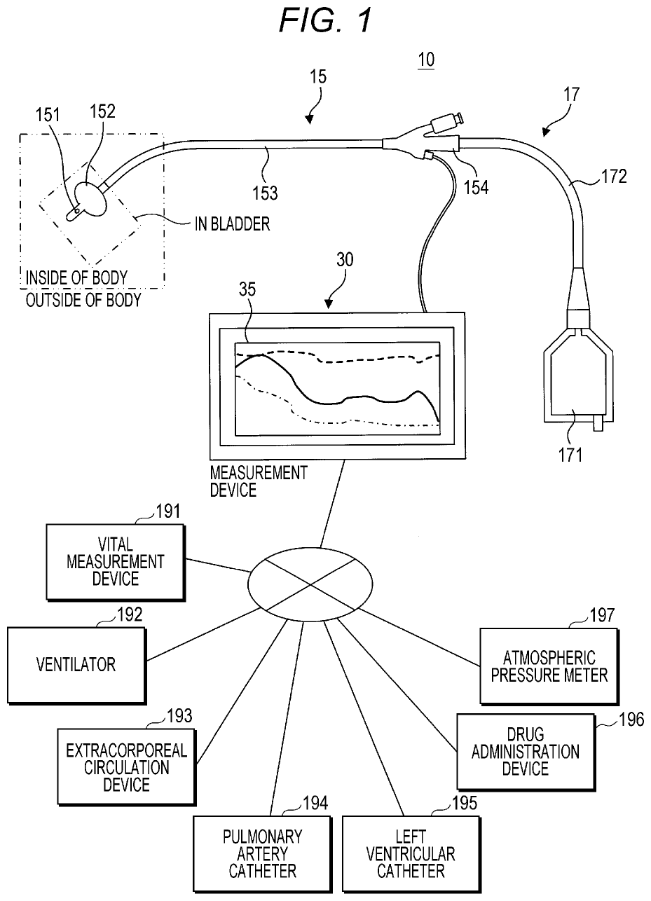

FIG. 1 is an explanatory view illustrating a configuration of a measurement system 10. The measurement system 10 can include a bladder indwelling catheter 15, a urine collection bag 17, and a measurement device 30. The measurement device 30 can be connected to various devices, for example, such as a vital measurement device 191, a ventilator 192, an extracorporeal circulation device 193, a pulmonary artery (PA) catheter 194, a left ventricular catheter 195, a drug administration device 196, or an atmospheric pressure meter 197 via a network such as a hospital information system (HIS) or an electric medical record (EMR). The measurement device 30 may be directly connected to other devices without a network.

The bladder indwelling catheter 15 includes a shaft 153 having a side hole 151 and a balloon 152 at a distal end, and a urination funnel 154 connected to one end of the shaft 153. The urine collection bag 17 includes a urine collection tube 172 and a bag 171 connected to one end of the urine collection tube 172. The other end of the urine collection tube 172 is connected to the urination funnel 154. The bladder indwelling catheter 15 may be a so-called integrated type that is supplied in a state of being undetachably connected (i.e., unable to be detached) to the urine collection bag 17.

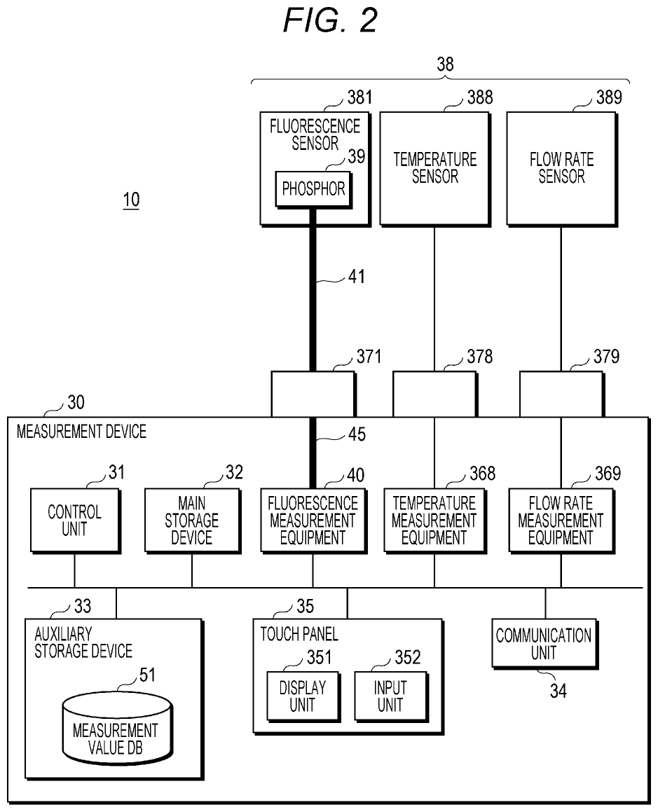

FIG. 2 is an explanatory view illustrating a configuration of the measurement system 10. The measurement system 10 includes a measurement device 30 and a sensor 38. In the present embodiment, the sensor 38 includes a fluorescence sensor 381, a temperature sensor 388, and a flow rate sensor 389. Each sensor 38 is attached to the bladder indwelling catheter 15 or the urine collection bag 17.

The fluorescence sensor 381 includes a phosphor 39 disposed so as to be able to contact urine flowing through a flow path from the side hole 151 to the bag 171, and optical components such as a lens and a filter. The measurement system 10 may include a sensor 38 other than the fluorescence sensor 381, the temperature sensor 388, and the flow rate sensor 389. The measurement system 10 may include a plurality of sensors 38 of the same type of sensors.

The measurement device 30 can include a control unit 31, a main storage device 32, an auxiliary storage device 33, a communication unit 34, a touch panel 35, temperature measurement equipment 368, a temperature sensor connector 378, flow rate measurement equipment 369, a flow rate sensor connector 379, fluorescence measurement equipment 40, a first connector 371, and a bus.

The control unit 31 is an arithmetic control device that executes a program of the present embodiment. For the control unit 31, one or a plurality of central processing units (CPUs), graphics processing units (GPUs), multi-core CPUs, or the like, can be used. The control unit 31 is connected to each hardware unit constituting the measurement device 30 via a bus.

The main storage device 32 is a storage device such as a static random access memory (SRAM), a dynamic random access memory (DRAM), or a flash memory. The main storage device 32 temporarily stores information necessary in the middle of processing to be performed by the control unit 31 and a program being executed by the control unit 31.

The auxiliary storage device 33 is a storage device such as an SRAM, a flash memory, a hard disk, or a magnetic tape. The measurement value database (DB) 51, a program to be executed by the control unit 31, and various data necessary for executing the program are stored in the auxiliary storage device 33. The communication unit 34 is an interface that performs communication between the measurement device 30 and a network or other devices. The measurement value DB 51 may be recorded in an external mass storage device connected to the measurement device 30.

The touch panel 35 can include a display unit 351 using, for example, a liquid crystal display panel or an organic electro-luminescence (EL) panel, and an input unit 352 stacked on the display unit 351. The touch panel 35 is attached to a chassis of the measurement device 30 as illustrated in FIG. 1. As illustrated in FIG. 1, a graph indicating various parameters measured using the sensor 38 in time series is displayed on the touch panel 35.

The touch panel 35 and the measurement device 30 may be separately provided. For example, a screen of equipment such as the vital measurement device 191 may also serve as the touch panel 35 of the measurement device 30. Instead of the touch panel 35, a combination of a display device using a liquid crystal display panel, an organic EL panel, or the like, and an input device such as a mouse, a keyboard, or a speech input device may be used.

The first connector 371 is an optical connector. An optical fiber 41 is connected between the first connector 371 and the phosphor 39. The phosphor 39 and the optical fiber 41 may be undetachably connected (i.e., unable to be detached) or may be detachably connected by an optical connector, or the like.

The temperature sensor connector 378 is a connector to which a cable connected to the temperature sensor 388 is connected. The flow rate sensor connector 379 is a connector to which a cable connected to the flow rate sensor 389 is connected. The temperature sensor 388 and the cable, and the flow rate sensor 389 and the cable may be undetachably connected (i.e., unable to be detached) or may be detachably connected by a connector, or the like. The fluorescence measurement equipment 40 and the first connector 371 are connected by a light guide path 45. Details of the fluorescence measurement equipment 40 will be described later.

The temperature measurement equipment 368 is connected to the temperature sensor 388 via the temperature sensor connector 378. In the present embodiment, the temperature sensor 388 is a thermocouple in which a temperature measurement contact is disposed near the side hole 151. The temperature measurement equipment 368 measures a temperature on the basis of a thermoelectromotive force generated in the thermocouple and outputs the temperature to the bus in real time. Temperature measurement using a thermocouple has been performed in related art, and thus, detailed description temperature measurement using a thermocouple will be omitted. In the following description, the temperature measured by the temperature measurement equipment 368 will be referred to as a temperature in the bladder.

For example, a temperature measurement contact may be disposed on the urination funnel 154 or the urine collection bag 17 in the middle of the shaft 153. In such a case, the temperature measurement equipment 368 measures a temperature of urine at a place where the temperature measurement contact is disposed instead of the temperature in the bladder. In the following description, the temperature of urine will be referred to as urine temperature.

The temperature sensor 388 is not limited to a thermocouple. Any sensor 38 available for temperature measurement can be utilized for the temperature sensor 388. For example, a thermistor, a resistance temperature detector, an integrated circuit (IC) temperature sensor, a fluorescent dye, or the like, can be used for the temperature sensor 388. In a case where the plurality of temperature sensors 388 is used, the temperature sensors 388 of the same type or different 388 types may be used.

The flow rate measurement equipment 369 is connected to the flow rate sensor 389 via the flow rate sensor connector 379. For example, the flow rate sensor 389 can be an optical flow rate sensor that transmits and receives a laser Doppler signal to and from urine, and the flow rate measurement equipment 369 is an optical flow rate meter. The flow rate measurement equipment 369 measures a flow rate of urine and outputs the flow rate to the bus in real time. In the following description, the flow rate of urine will be referred to as a urine flow rate. The flow rate measurement equipment 369 is an example of a flow rate measurement unit.

Principle of the flow rate measurement is not limited to optical flow rate measurement. Thus, the temperature sensor 388 is not limited to the optical flow rate sensor. The temperature sensor 388 may be, for example, an ultrasonic flow rate sensor or a thermal flow rate sensor.

The flow rate measurement equipment 369 may measure the urine flow rate by acquiring change over time in the weight of the bag 171 from a balance that measures the weight of the bag 171. A structure that drops a liquid and a sensor 38 that counts droplets may be disposed in the flow path of the urine collection bag 17. The flow rate measurement equipment 369 can measure the flow rate on the basis of the counted number of the droplets and a volume of the droplets.

A form of the optical fiber 41 that connects the sensor 38 and the measurement device 30 and a wire such as an electric wire is not limited to a form as illustrated in FIG. 2 in which the optical fiber 41 and the wire are connected to the first connector 371, the temperature sensor connector 378, and the flow rate sensor connector 379 which are independent of each other. Instead of the first connector 371, the temperature sensor connector 378, and the flow rate sensor connector 379, a composite connector in which the functions of these three connectors are integrated may be provided.

In a case where the composite connector is provided, a composite cable in which the optical fiber 41 and a wire material such as an electric wire are bundled may be used. In other words, the bladder indwelling catheter 15 and the urine collection bag 17 may be connected to the measurement device 30 by one cable and one composite connector in appearance.

The temperature sensor connector 378 and the temperature sensor 388 may be integrally configured, and the measured temperature may be transmitted to the measurement device 30 wirelessly. Similarly, the flow rate sensor 389 and the flow rate measurement equipment 369 may be integrally configured, and the measured flow rate may be transmitted wirelessly to the measurement device 30.

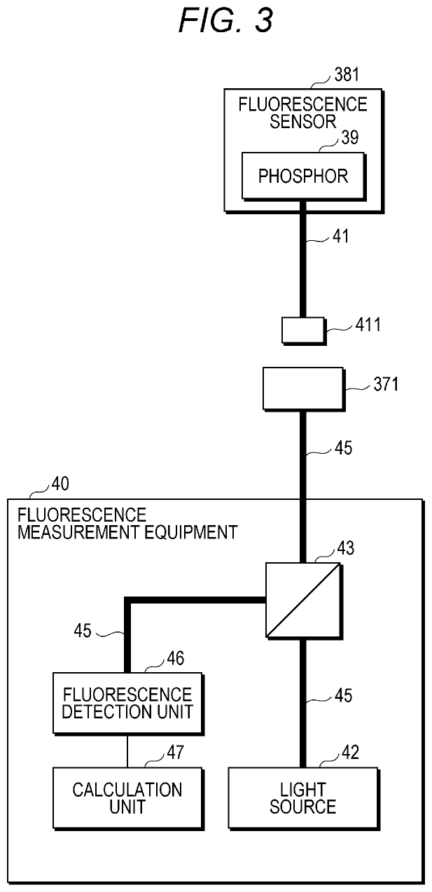

FIG. 3 is an explanatory view illustrating a configuration of the fluorescence measurement equipment 40. The fluorescence measurement equipment 40 can include a light source 42, a beam splitter 43, a fluorescence detection unit 46, and a calculation unit 47. The light guide path 45 connects between the light source 42 and the beam splitter 43, between the beam splitter 43 and the fluorescence detection unit 46, and between the beam splitter 43 and the first connector 371. An optical fiber connector 411 connectable to the first connector 371 is disposed at an end portion of the optical fiber 41.

The light source 42 can be, for example, a light emitting diode (LED) or a laser diode. The light source 42 emits excitation light that excites the phosphor 39. The light emitted from the light source 42 includes a part of the wavelength of fluorescence emitted from the phosphor 39.

In a case where the light emitted from the light source 42 includes the wavelength of the fluorescence emitted from the phosphor 39, or in a case where it is desirable to set a wavelength of the excitation light to an accurate excitation wavelength, the optical filter may be disposed in an optical path of the light guide path 45.

The excitation light emitted from the light source 42 irradiates the phosphor 39 via the light guide path 45, the beam splitter 43, and the optical fiber 41. In a case where the phosphor 39 is in contact with urine, fluorescence corresponding to an oxygen partial pressure or an oxygen concentration in the urine is emitted. In the following description, the oxygen partial pressure in urine will be referred to as a urinary oxygen partial pressure.

The fluorescence is incident on the beam splitter 43 via the optical fiber 41 and the light guide path 45. The fluorescence is incident on the light guide path 45 connected to the fluorescence detection unit 46 by the beam splitter 43. The fluorescence detection unit 46 can include, for example, a photoelectric conversion element such as a photodiode and converts fluorescence into an electrical signal. The calculation unit 47 analyzes the electrical signal and outputs the urinary oxygen partial pressure to the bus in real time. The calculation unit 47 is an example of a measurement unit that sequentially measures parameters related to the urine on the basis of information obtained by the sensor 38.

The first connector 371, the temperature sensor connector 378, and the flow rate sensor connector 379 function as a sensor data acquisition unit that acquires sensor data from the sensor 38. The calculation unit 47, the temperature measurement equipment 368, and the flow rate measurement equipment 369 function as a calculation unit that sequentially calculates parameters related to the urine using the sensor data.

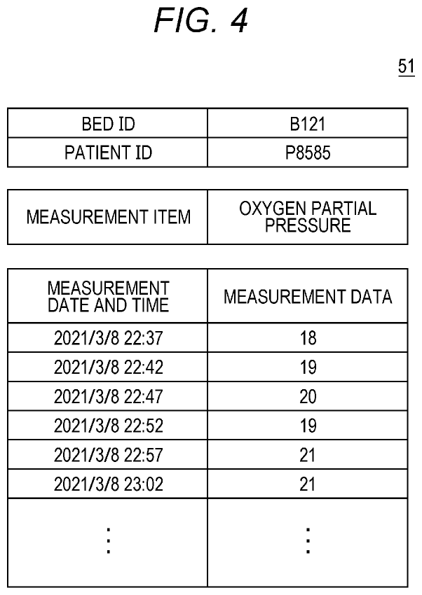

FIG. 4 is an explanatory view for explaining record layout of the measurement value DB 51. The measurement value DB 51 is a database (DB) that records a bed identifier (ID), a patient ID, a measurement item, measurement date and time, and measurement data in association with each other.

The measurement value DB 51 can have a bed ID field, a patient ID field, a measurement item field, a measurement date and time field, and a measurement data field. A bed ID uniquely assigned to a bed is recorded in the bed ID field. In the patient ID field, a patient ID uniquely assigned to a patient is recorded.

In the measurement item field, measurement items are recorded. The measurement items are, for example, an “oxygen partial pressure”, a “temperature”, and a “flow rate”. Measurement date and time is recorded in the measurement date and time field. In the measurement data field, measurement data measured using the sensor 38 is recorded.

In FIG. 4, in the measurement date and time field and the measurement data field, the date and time when the urinary oxygen partial pressure is measured by the fluorescence measurement equipment 40 and the urinary oxygen partial pressure are recorded, respectively. In the measurement date and time field and the measurement data field in a case where the measurement item field is a “temperature”, the date and time when the temperature in the bladder is measured by the temperature measurement equipment 368 and the temperature in the bladder are recorded, respectively.

In the measurement date and time field and the measurement data field in a case where the measurement item field is a “flow rate”, the date and time when the flow rate of urine is measured by the flow rate measurement equipment 369 and the flow rate of urine are recorded, respectively. The measurement value DB 51 has one record for one measurement of one item.

As described above, each measurement item, the measurement date and time, and the measurement data are recorded in association with each other. Thus, even in a case where a measurement interval differs for each measurement item, the control unit 31 can record the measurement data in the measurement value DB 51.

Outline of a method of using the measurement system 10 of the present embodiment will be described. A user such as a doctor inserts the shaft 153 into the urethra of the patient. In a state where a distal end of the shaft 153 enters the inside of the bladder, the user inflates the balloon 152. The balloon 152 illustrated in FIG. 1 is in an inflated state. By inflating the balloon 152, the shaft 153 does not come out of the urethra. The urine of the patient is conducted through a flow path connecting the side hole 151 and the bag 171 and is accumulated in the bag 171.

The urinary oxygen partial pressure is output from the fluorescence measurement equipment 40, the temperature in the bladder is output from the temperature measurement equipment 368, and the urine flow rate is output from the flow rate measurement equipment 369 to the bus. The urinary oxygen partial pressure, the urine flow rate and the temperature in the bladder are exemplary parameters related to urine. The control unit 31 records each data in the measurement value DB 51 in association with the measurement date and time and displays the data on the touch panel 35 in a form of a graph using a time axis as illustrated in FIG. 1. The graph of FIG. 1 indicates time on a horizontal axis and indicates the urinary oxygen partial pressure, the urine flow rate, and the temperature in the bladder on a vertical axis.

The user can rather easily understand changing states of the parameters from the graph displayed on the touch panel 35. For example, in a case where it is desired to confirm long-term trend or change in the latest short period, the user can relatively easily change the horizontal axis of the graph by operating the touch panel 35. Details of the screen display and details of the operation to be performed by the user will be described later.

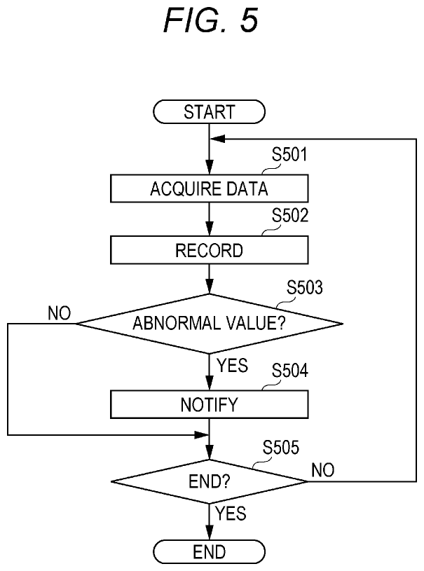

FIG. 5 is a flowchart for explaining flow of processing of the program. In the program illustrated in FIG. 5, the control unit 31 starts the program of FIG. 5 in a case where the user gives an instruction to start measurement.

The control unit 31 acquires data output from the fluorescence measurement equipment 40, the temperature measurement equipment 368, or the flow rate measurement equipment 369 to the bus (S501). The control unit 31 creates a new record in the measurement value DB 51 and records the acquired data (S502). The control unit 31 determines whether or not the acquired data indicates an abnormal value (S503).

A criterion for determining whether or not the value is an abnormal value is determined in advance for each measurement item. For example, in a case where each piece of data is not included in a predetermined normal range, the control unit 31 determines that the value is an abnormal value. The control unit 31 may determine whether or not the value is an abnormal value on the basis of a change rate of each measurement item. The control unit 31 may determine whether or not the value is an abnormal value on the basis of a combination of a plurality of measurement items.

In a case where it is determined that the value is an abnormal value (YES in S503), the control unit 31 makes a notification that an abnormal value has been detected (S504). The notification can be made, for example, by display on the touch panel 35 or sound output from the measurement device 30. The control unit 31 may transmit the notification, for example, to a nurse station, an electronic medical record system, or the like, via a network such as HIS.

In a case where it is determined that the value is not an abnormal value (NO in S503), or after the processing of S504 is finished, the control unit 31 determines whether or not to end the processing (S505). For example, in a case where an end instruction is received from the user, the control unit 31 determines to end the processing.

In a case where it is determined not to end the processing (NO in S505), the processing of the control unit 31 returns to S501. In a case where it is determined to end the processing (YES in S505), the control unit 31 ends the processing.

Between S501 and S502, the control unit 31 may correct the oxygen partial pressure in the urine calculated by the calculation unit 47 on the basis of the atmospheric pressure, the pressure in the bladder, the temperature, or the chloride concentration in the urine. In such a case, the control unit 31 implements a function of a correction unit that corrects the oxygen partial pressure in the urine.

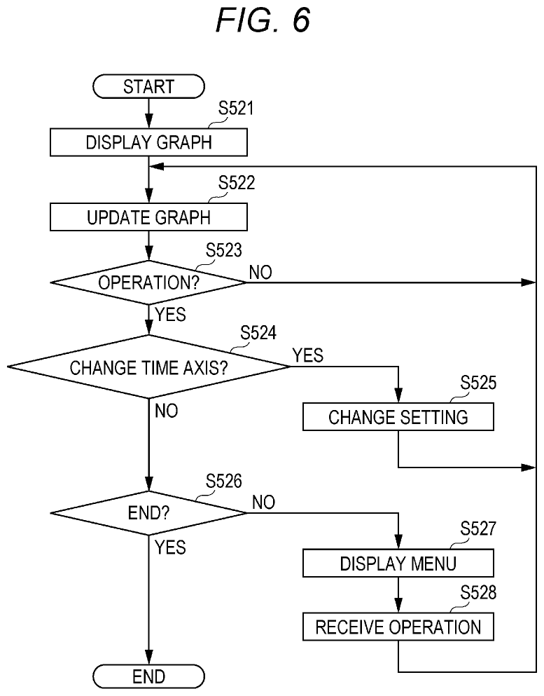

FIG. 6 is a flowchart for explaining flow of processing of the program. The control unit 31 executes the program of FIG. 6 in parallel while executing the program described with reference to FIG. 5.

The control unit 31 displays a graph on the touch panel 35 (S521). In S521, only frames of the graph such as the vertical axis and the horizontal axis are displayed. Initial settings of the vertical axis, the horizontal axis, display items, and the like, are stored in advance in the main storage device 32 or the auxiliary storage device 33.

The control unit 31 reads data from the measurement value DB 51 and updates the graph (S522). While the program of FIG. 6 is being executed, data is sequentially added to the measurement value DB 51 by the program described with reference to FIG. 5.

The control unit 31 determines whether or not operation by the user has been received (S523). In a case where it is determined that operation has not been received (NO in S523), the processing of the control unit 31 returns to S522. In a case where it is determined that operation has been received (YES in S523), the control unit 31 determines whether or not the operation is operation of changing the time axis (S524). A specific example of the operation of changing the time axis will be described later.

In a case where it is determined that the operation is operation of changing the time axis (YES in S524), the control unit 31 changes the setting of the time axis (S525). Thereafter, the processing of the control unit 31 returns to S522. In subsequent S522, the control unit 31 displays a graph in which the time axis is changed.

In a case where it is determined that the operation is not the operation of changing the time axis (NO in S524), the control unit 31 determines whether or not the operation is operation of ending the program (S526). In a case where it is determined that the operation is operation of ending the program (YES in S526), the control unit 31 ends the processing. The control unit 31 may end the processing of the program described with reference to FIG. 5 together with the program illustrated in FIG. 6.

In a case where it is determined that the operation is not operation of ending the program (NO in S526), the control unit 31 displays a menu screen on the touch panel 35 (S527). The control unit 31 receives operation by the user via the touch panel 35 (S528). Thereafter, the processing of the control unit 31 returns to S522.

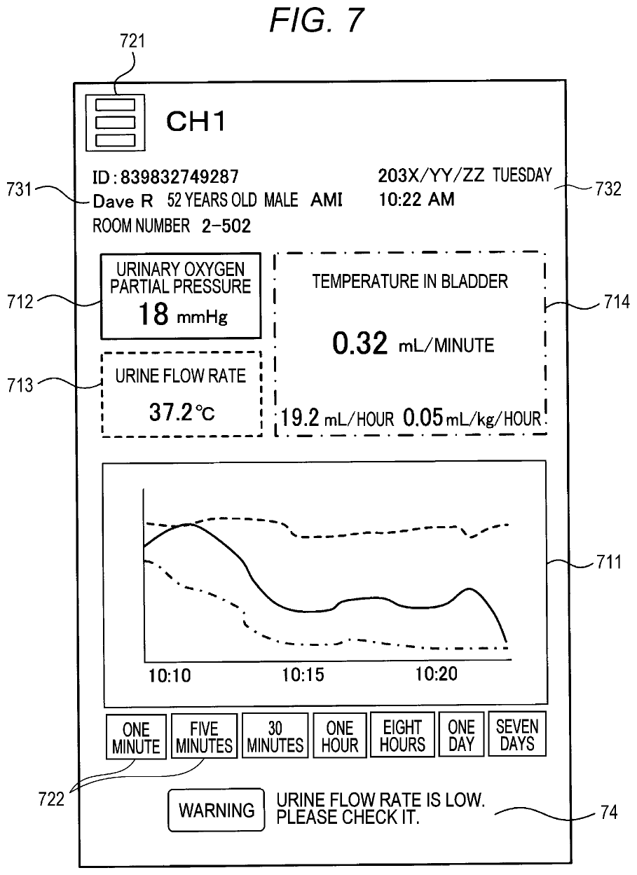

FIGS. 7 to 12 are explanatory views for explaining an example of the display screen. FIG. 7 illustrates an example of the screen displayed on the touch panel 35 by the control unit 31 in S521. A menu display button 721, a patient information field 731, a date and time field 732, an oxygen partial pressure field 712, a temperature field 713, a urine flow rate field 714, a graph field 711, a time axis change button 722, and a notification field 74 are displayed on the screen from the upper left to the lower right.

The menu display button 721 can be a button for accepting an instruction to transition to the menu screen to be described next. In the patient information field 731, a patient ID, patient name, age, disease name, and a room number are displayed.

The current date and time is displayed in the date and time field 732. In the oxygen partial pressure field 712, the latest measurement value of the urinary oxygen partial pressure is displayed. The temperature field 713 displays the latest measurement value of the temperature in the bladder. In the urine flow rate field 714, the latest measurement value of the urine flow rate is displayed.

In the graph field 711, a graph indicating the measurement values recorded in the measurement value DB 51 in time series is displayed. As described above, the graph indicates time on the horizontal axis and indicates the urinary oxygen partial pressure, the urine flow rate, and the temperature in the bladder on the vertical axis. In FIG. 7, a scale on the vertical axis is not illustrated.

The graph indicates the urinary oxygen partial pressure with a solid line. A frame surrounding the oxygen partial pressure field 712 is also indicated with a solid line. The graph indicates the temperature in the bladder with a broken line. A frame surrounding the temperature field 713 is also indicated with a broken line. The graph indicates the urine flow rate with an alternate long and short dash line. A frame surrounding the urine flow rate field 714 is also indicated with an alternate long and short dash line. Line types of the frames surrounding the oxygen partial pressure field 712, the temperature field 713, and the urine flow rate field 714 serve as a legend field, so that the user can relatively easily grasp (or understand) what the graph indicates. Numbers indicating the oxygen partial pressure, the temperature in the bladder and the urine flow rate may each be the same as the color of the graph. The urinary oxygen partial pressure may be displayed by using an oxygen concentration in urine. The control unit 31 performs conversion from the oxygen partial pressure to the oxygen concentration on the basis of data such as the temperature and the atmospheric pressure which are simultaneously measured.

The time axis change button 722 is a button for receiving change of the time axis. In FIG. 7, “5 minutes” is selected, and one scale of the graph field 711 is 5 minutes. For example, in a case where the user selects “1 minute”, the control unit 31 changes one scale of the graph field 711 to 1 minute. The time axis change button 722 is an example of a reception unit that receives a change instruction related to the time axis.

A slider may be displayed instead of the time axis change button 722. In a case where the user performs operation of moving the slider, the control unit 31 changes the time axis of the graph field 711. The control unit 31 may change the time axis in a case where the user performs tap operation on the graph field 711. In addition, a user interface can be used instead of the time axis change button 722.

The notification field 74 in FIG. 7 includes a message such as “urine output is small. Please check it”. This notification is a notification output by the control unit 31 in S504 in a case where it is determined that the urine flow rate is less than a threshold in S503 of the flowchart described using FIG. 5.

In the notification field 74, a notification based on data measured by the measurement device 30, such as the urinary oxygen partial pressure or the urine temperature, may be displayed. In the notification field 74, a notification based on information acquired from other devices such as the vital measurement device 191 or the ventilator 192 and data analyzed by the measurement device 30 may be displayed.

The control unit 31 may display a moving average of the measurement data of each measurement item in the graph field 711. In a case where noise associated with body motion, or the like, of the patient is likely to be superimposed on the measurement data, the moving average is displayed, so that the user can appropriately grasp (or understand) the patient's condition.

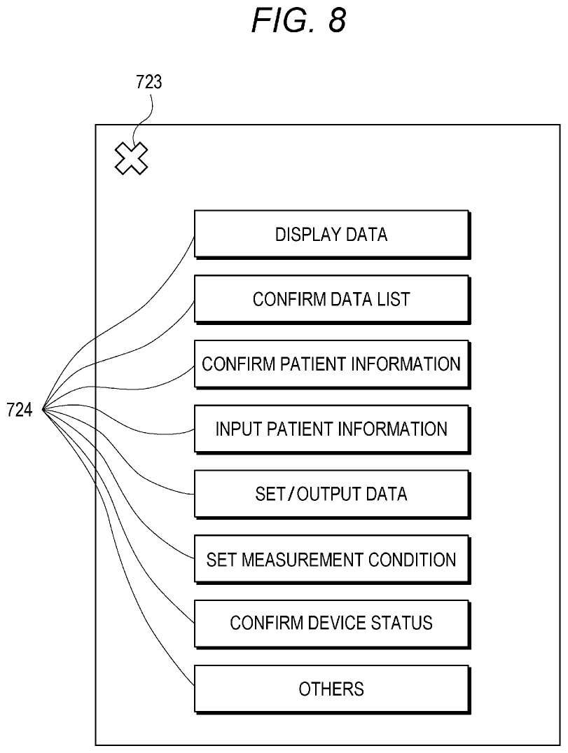

FIG. 8 illustrates an example of the menu screen to be displayed by the control unit 31 in a case where the user selects the menu display button 721. The menu selection buttons 724 of “display data”, “confirm data list”, “confirm patient information”, “input patient information”, “set/output data”, “set measurement condition”, “confirm device status”, and “others” are displayed. The control unit 31 receives selection of the menu selection button 724 by the user. In a case where selection of the menu selection button 724 of “data display” is received, the control unit 31 returns the screen to the screen described with reference to FIG. 7.

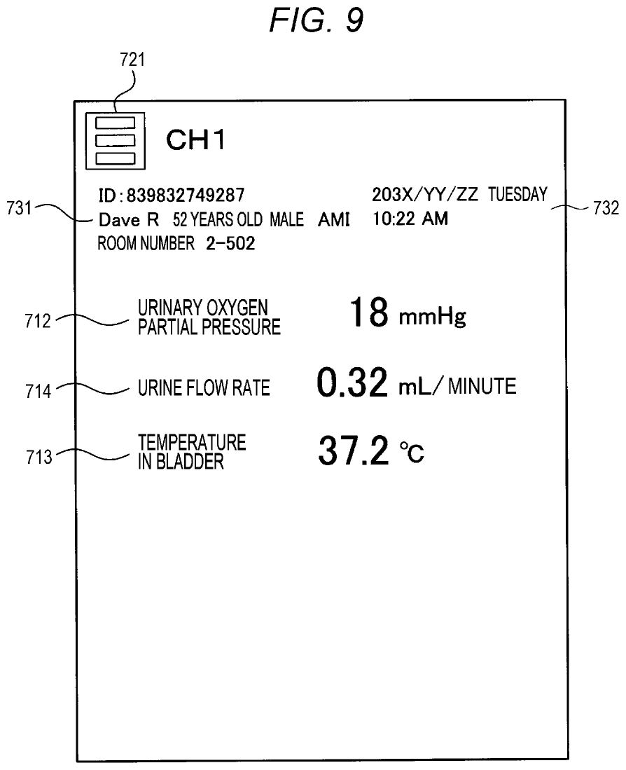

FIG. 9 illustrates an example of the screen to be displayed by the control unit 31 in a case where selection of the menu selection button 724 of “confirm data list” is received. The menu display button 721, the patient information field 731, the date and time field 732, the oxygen partial pressure field 712, the urine flow rate field 714, and the temperature field 713 are displayed on the screen from the upper left to the lower right.

In the patient information field 731 of FIG. 9, height and weight are displayed in addition to the patient ID, the patient name, the age, the disease name, and the room number. The other items are similar to those of the screen described with reference to FIG. 7, and thus, the description of the other items is omitted. The same applies to the following description of the screen.

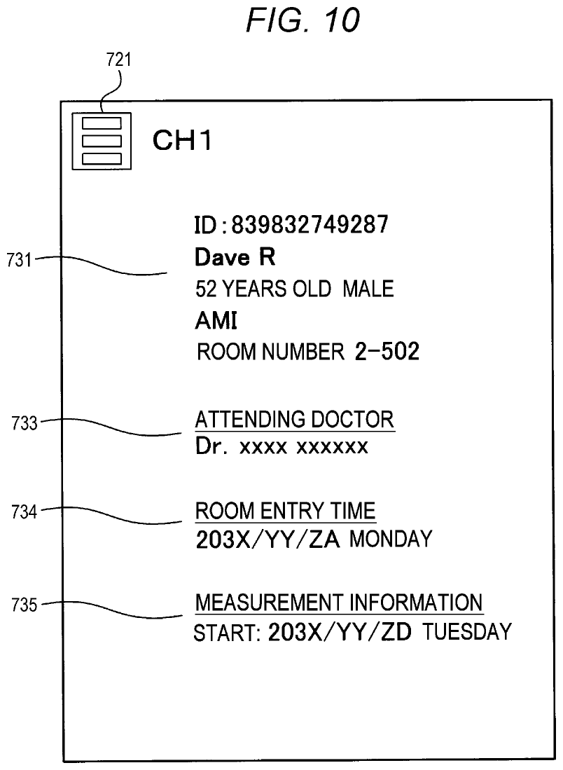

FIG. 10 illustrates an example of the screen to be displayed by the control unit 31 in a case where selection of the menu selection button 724 of “confirm patient information” is received. On the screen, the menu display button 721, the patient information field 731, an attending doctor field 733, a room entry time field 734, and a measurement information field 735 are displayed from top to bottom.

In the attending doctor field 733, name of the attending doctor in charge of the patient is displayed. A communication button for contacting the attending doctor may be arranged in the vicinity of the attending doctor field 733. For example, a contact address of the attending doctor such as a mobile phone number may be displayed in the vicinity of the attending doctor field 733.

In the room entry time field 734, time when the patient enters the room displayed in the patient information field 731 is displayed. In the measurement information field 735, time when measurement is started using the measurement device 30 is displayed.

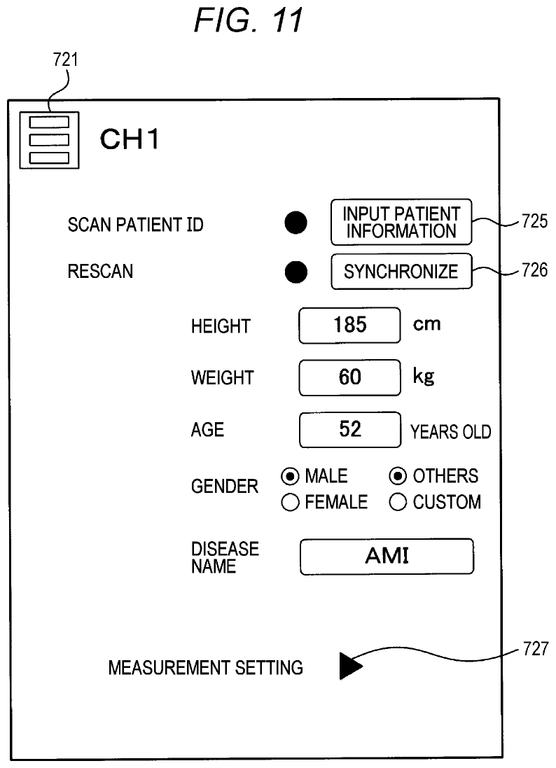

FIG. 11 illustrates an example of the screen to be displayed by the control unit 31 in a case where selection of the menu selection button 724 of “input patient information” is received. The menu display button 721, an ID read button 725, and a patient information acquisition button 726 are displayed at the top of the screen. The ID read button 725 is used to read a patient ID written on a wristband worn by a patient, or the like, for example, using a barcode reader. A black circle on the left side of the ID read button 725 indicates that reading of the patient ID is completed. The user can redo reading of the patient ID using the read ID button 725.

The control unit 31 may receive an input of the patient ID via an input device such as a keyboard. Procedure for reading the patient ID varies depending on a medical institution. It is desirable that the measurement device 30 can set processing according to procedure of each medical institution.

In a case where the patient information acquisition button 726 is selected, the control unit 31 synchronizes the information recorded in the control unit 31 with the information recorded in the electronic medical record, or the like, by acquiring patient data such as the name of the patient from the electronic medical record, or the like, using the patient ID as a key. In FIG. 11, the control unit 31 displays the height, the weight, the age, the gender, and the disease name of the patient. The control unit 31 may receive correction of these items via the screen illustrated in FIG. 11. A connection equipment selection button 727 is displayed at the bottom of the screen illustrated in FIG. 11.

The items illustrated in FIG. 11 are examples of the patient data. The patient data may include patient background factors such as a body mass index (BMI) calculated from the height and the weight in addition to the height, the weight, the age, the gender, and the disease name illustrated in FIG. 11. The patient data may include data on various examination items such as serum creatinine values, urea nitrogen amounts, and the like.

The control unit 31 implements the function of a weight acquisition unit that acquires the weight of the patient via the item of the weight on the screen of FIG. 11. The weight acquisition unit is an example of a patient information acquisition unit. The control unit 31 may acquire the weight of the patient via a weight scale having a communication function. The control unit 31 may obtain patient data from a system such as HIS or EMR.

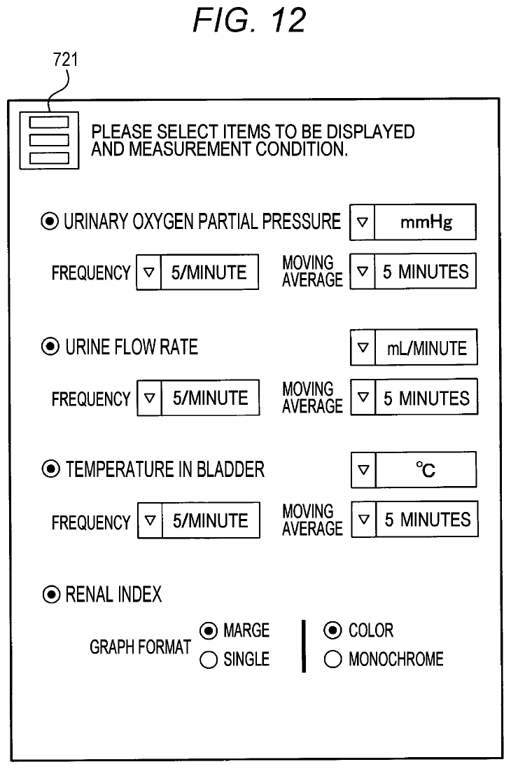

FIG. 12 illustrates an example of the screen to be displayed by the control unit 31 in a case where selection of the menu selection button 724 of “set measurement condition” is received or in a case where selection of the connection equipment selection button 727 is received. The menu display button 721 is displayed at the upper left of the screen.

The control unit 31 receives setting of measurement conditions such as units of the urinary oxygen partial pressure, the urine flow rate, and the temperature in the bladder, a measurement frequency, and a section to be used at the time of moving average calculation. Furthermore, the control unit 31 receives setting of whether or not a renal index is calculated, a graph format, and screen display color. The renal index will be described later.

The user does not have to change settings such as the measurement frequency and the moving average. In such a case, a pull-down menu indicated by an inverted triangle is not displayed on the right side of a setting value of each item. The control unit 31 may accept setting changes of the measurement frequency, the moving average, and the like, only in a case where a specific user such as a person in charge of management of the measurement device 30 uses the device.

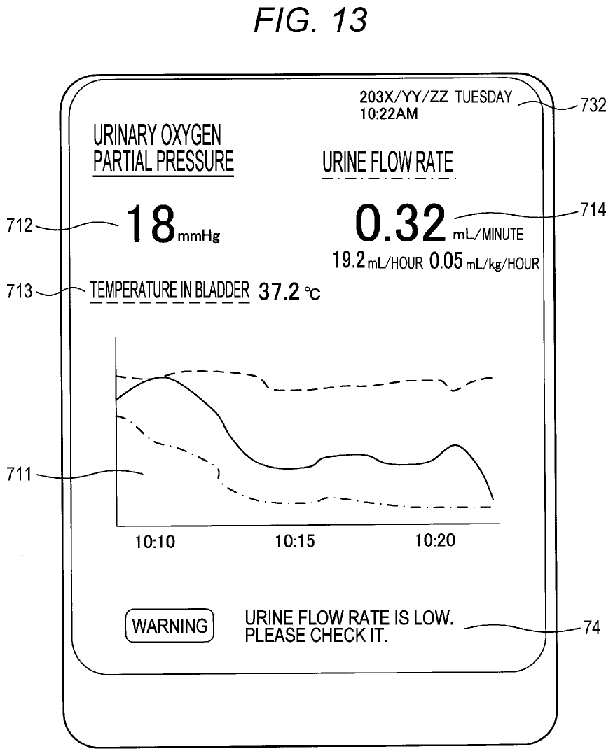

FIGS. 13 to 15 are explanatory views illustrating modifications of the display screen. The control unit 31 may display the screens illustrated in FIGS. 13 to 15 instead of the screen described with reference to FIG. 7. The user may be able to switch the display format of the screen.

In the screen illustrated in FIG. 13, the patient information field 731 and the time axis change button 722 are not displayed. The number of items to be displayed is relatively small, and thus, the display screen can be simple, so that the user can relatively quickly confirm necessary information.

For example, the control unit 31 changes the time axis in a case where tap operation, flick operation, or the like, in the vicinity of the horizontal axis of the graph field 711 is received. The user may be able to set user's operation to change the time axis. The control unit 31 may receive an instruction to change the time axis by the user via speech input.

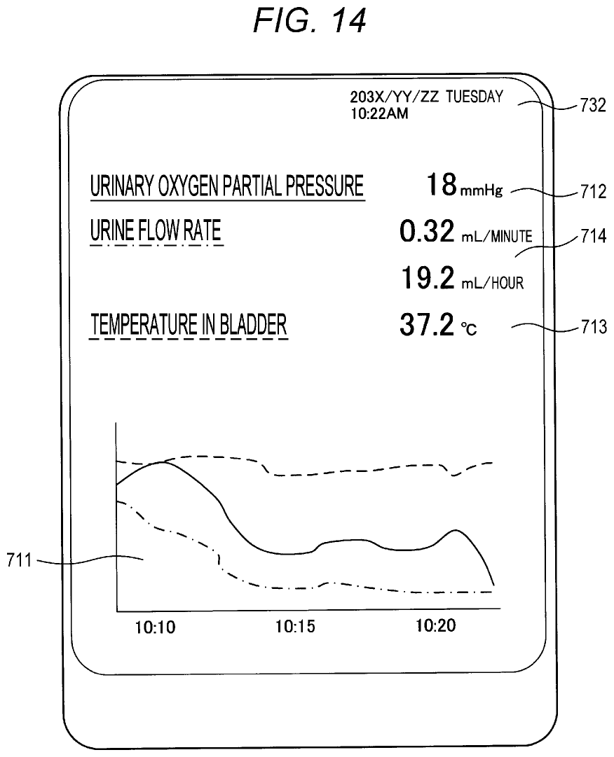

In the screen illustrated in FIG. 14, the oxygen partial pressure field 712, the urine flow rate field 714, and the temperature field 713 are arranged in a vertical line with characters of the same size. The display screen can be simpler than that in FIG. 13.

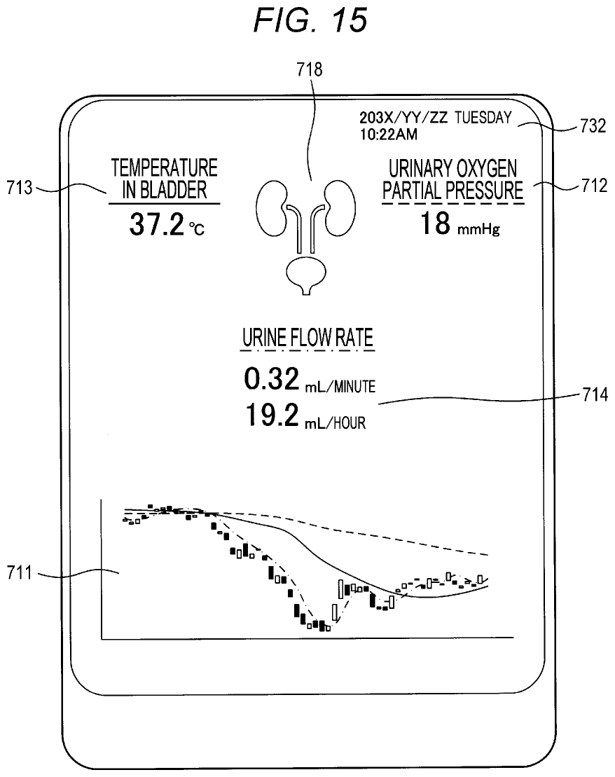

On the screen illustrated in FIG. 15, the oxygen partial pressure field 712, the temperature field 713, and the urine flow rate field 714 are displayed with the kidney icon 718 schematically illustrating a shape of the kidney and the bladder as a center.

Data for about two weeks is displayed in the graph field 711. The urinary oxygen partial pressure indicated by the solid line, the temperature in the bladder indicated by the broken line, and the urine flow rate indicated by the alternate long and short dash line are each displayed as a moving average in units of several days. The urine flow rate can also be displayed in a form of a so-called candlestick chart indicating the lowest, highest, first and last values of a day. The candlestick chart has been used in related art for displaying price movements of stock prices, and the like, and thus, the detailed description of the candlestick chart will be omitted.

The candlestick chart may also be displayed at the urinary oxygen partial pressure indicated by the solid line and the temperature inside the bladder indicated by the broken line. It is desirable that the user can relatively easily switch whether or not to display the candlestick chart. The user can rather easily understand changing trends of each piece of measurement data from the candlestick chart.

The display of the graph field 711 is an example. The items displayed in the graph field 711 are not limited to the three items of the urinary oxygen partial pressure, the temperature in the bladder, and the urine flow rate. It is desirable that the user can appropriately set the items and the display format illustrated in the graph field 711.

In a case where there is an item indicating an abnormal value, the control unit 31 may change the display to display that rather easily attracts the user's attention, such as displaying the kidney icon 718 in red or blinking.

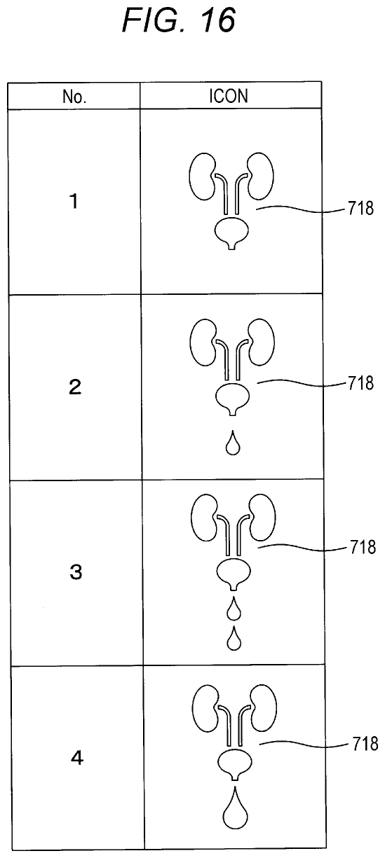

FIG. 16 is an explanatory view for explaining a modification of the kidney icon 718. The control unit 31 selects and displays the kidney icon 718 according to the urine flow rate. No. 1 is a kidney icon 718 to be used in a case where the urine flow rate is very low, and No. 4 is a kidney icon 718 to be used in a case where the urine flow rate is very high. The control unit 31 may display a portion of a drop falling from the bladder as an animation. Not only the user who is a medical professional but also a visiting customer and the patient can intuitively understand change in the urine flow rate.

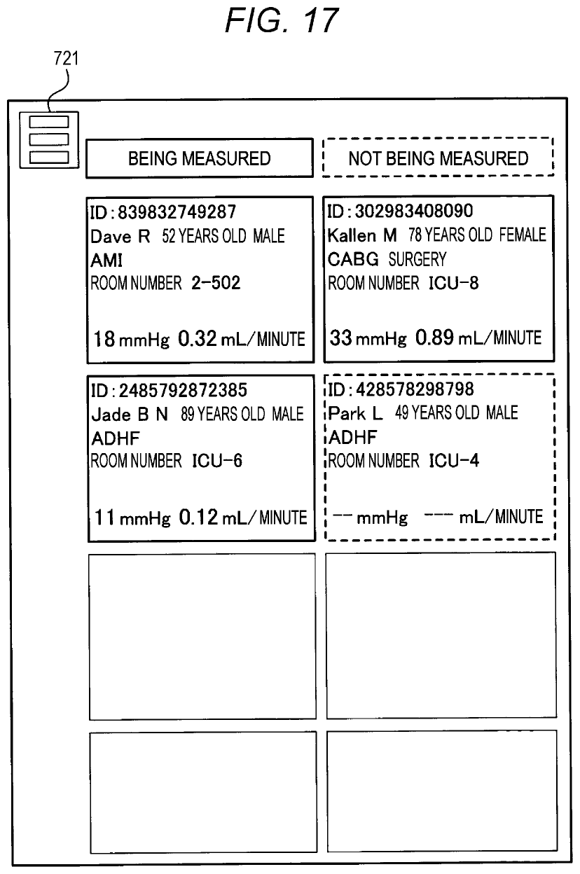

FIG. 17 is an explanatory view illustrating a modification of the display screen. FIG. 17 is a screen example when a plurality of devices is managed by one device. Data measured by the plurality of measurement systems 10 is listed in a so-called tile format. A user such as a nurse can check information of many patients at once. In this event, it is possible to understand a condition of each patient at a glance by expressing states of numerical values, and the like, with color or a line type of an edge during measurement, while measurement is stopped, or the like.

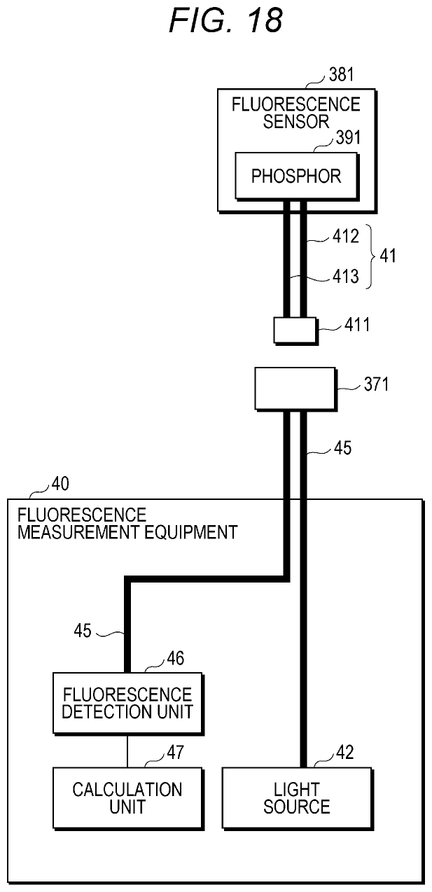

FIG. 18 is an explanatory view for explaining a modification of the fluorescence measurement equipment 40. In the present modification, the light guide path 45 directly connects between the light source 42 and the first connector 371 and between the fluorescence detection unit 46 and the first connector 371. The optical fiber connector 411 and the phosphor 39 are connected by a fiber for irradiation 412 and a fiber for light reception 413.

Through the optical fiber connector 411 and the first connector 371, the fiber for irradiation 412 is connected to the light guide path 45 connected to the light source 42, and the fiber for light reception 413 is connected to the light guide path 45 connected to the fluorescence detection unit 46.

The excitation light emitted from the light source 42 irradiates the phosphor 39 via the light guide path 45 and the fiber for irradiation 412. In a case where the phosphor 39 comes into contact with urine, fluorescence corresponding to a concentration of the substance to be measured in the urine is emitted. In the fluorescence using oxygen as a quencher, fluorescence corresponding to the oxygen concentration and the oxygen partial pressure is emitted.

The fluorescence is incident on the fluorescence detection unit 46 via the fiber for light reception 413 and the light guide path 45. The fluorescence detection unit 46 converts fluorescence into an electrical signal. The calculation unit 47 analyzes the electrical signal and outputs the urinary oxygen partial pressure or the urinary oxygen concentration to the bus in real time. According to the present modification, it is possible to provide the measurement system 10 that does not use the beam splitter 43.

The fiber for irradiation 412 and the fiber for light reception 413 may be bundled to form the optical fiber 41. Note that the optical fiber 41 can be a bundle of relatively thin fiber wires. The number of fiber wires included in the fiber for irradiation 412 and the number of fiber wires included in the fiber for light reception 413 may be the same or different. In the sensor 38 using fluorescence, intensity of fluorescence is smaller than intensity of excitation light, so that it is desirable to increase the number of fiber wires included in the fiber for light reception 413 to increase light reception efficiency.

The optical fiber connector 411 and the first connector 371 may be separated into for irradiation of excitation light and for reception of fluorescence, respectively. In such a case, the number of the optical fibers 41 is one on the side of the phosphor 39, and the optical fibers 41 are branched into two on the side of the optical fiber connector 411, and the optical fiber connector 411 is attached to each end. A coupling may be provided at a branch portion of the optical fibers 41.

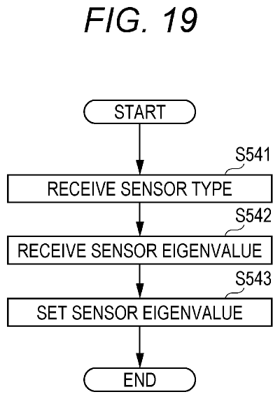

FIG. 19 is a flowchart for explaining flow of processing of the program of the modification. The program illustrated in FIG. 19 is a program that enables use of a sensor eigenvalue determined for each sensor 38.

The sensor eigenvalue is a value acquired for each sensor 38 at the time of manufacturing the sensor 38 and is used for calculation, correction, or the like, of a measurement result. The sensor eigenvalue is described, for example, in a packaging material of the sensor 38. Possible values to be used can include a slope of a regression line measured under certain conditions, a phase angle according to the oxygen concentration, a value related to decay time or the intensity, and the like. For the correction, correction regarding a temperature, a pressure, and chloride ions may be performed, and an actual value may be used, or a previously-reported mathematical expression used for calibration may be used. In addition, it is also possible to perform correction using a numerical value obtained by manually inputting necessary information separately by the user.

The control unit 31 receives an input of a sensor type via the input unit 352 (S541). The control unit 31 receives an input of a sensor eigenvalue via the input unit 352 (S542). By S542, the control unit 31 implements a function of a sensor eigenvalue acquisition unit that acquires a sensor eigenvalue.

The control unit 31 sets the sensor eigenvalue received in S542 to the measurement equipment corresponding to the sensor type received in S541 (S543). Thereafter, the measurement equipment outputs measurement data measured using the sensor eigenvalue. The measurement equipment implements a function of a calibration unit that calibrates the sensor 38 on the basis of the sensor eigenvalue.

The fluorescence measurement equipment 40 may have a function of calibrating the sensor 38. For example, environments for calibration in which target concentration is known are prepared for two types of target concentration. In both environments, the sensor 38 can be calibrated by measuring the sensor value output from each sensor 38. A calibration method of the sensor 38 is appropriately determined for each measurement system 10.

According to the present embodiment, it is possible to provide the measurement system 10 that displays parameters measured using the bladder indwelling catheter 15 in a graph using a time axis. According to the present embodiment, it is possible to provide the measurement system 10 in which the user can relatively easily change the time axis of the displayed graph.

The phosphor 39 may emit fluorescence in response to carbon dioxide in the urine. By analyzing characteristics of the fluorescence, a partial pressure of carbon dioxide in the urine can be measured in real time. Examples of the characteristics of the fluorescence to be used here can include intensity, decay time, or a phase angle of the fluorescence. A light emitting state of the phosphor 39 may be changed by a hydrogen ion index of the urine. By analyzing the characteristics of the fluorescence, the hydrogen ion index of the urine, that is, a potential of hydrogen (pH) can be measured in real time. The characteristics of the fluorescence to be used here can include intensity, decay time, a phase angle, and the like.

The phosphor 39 may emit fluorescence in response to a concentration of any component such as potassium, sodium, creatinine, or urea nitrogen in the urine. By analyzing the characteristics of the fluorescence, a concentration of the component contained in the urine, a creatinine value, or a urea nitrogen amount can be measured in real time. In addition, the phosphor 39 that reacts with an arbitrary component in the urine to emit fluorescence may be used.

The light emitting state of the phosphor 39 may change depending on the temperature. By analyzing the characteristics of the fluorescence, the temperature of the urine can be measured in real time. In other words, the phosphor 39 may be used for the temperature sensor 388.

The light emitting state of the phosphor 39 may be changed depending on the flow rate of the urine to be contacted. By analyzing the fluorescence, the flow rate of the urine can be measured in real time. In other words, the phosphor 39 may be used for the flow rate sensor 389.

The light emitting state of the phosphor 39 may be changed depending on the pressure. For example, an intravesical pressure can be measured in real time by placing the phosphor 39 in the bladder and analyzing the fluorescence. In other words, the phosphor 39 may be used in an intravesical pressure sensor.

The measurement system 10 may include a sensor 38 capable of measuring a perfusion state around the urethra. Examples of the sensor 38 include a sensor 38 that analyzes an absorption wavelength specific to oxyhemoglobin. The measurement system 10 may include two or more fluorescence sensors 381 having the phosphor 39 reactive to oxygen in urine and two or more temperature sensors 388. The fluorescence sensor 381 including the phosphor 39 that reacts with oxygen in urine is an example of the oxygen sensor of the present embodiment. The measurement system 10 may include a sensor 38 capable of measuring a blood flow velocity around the urethra.

The measurement device 30 may include a second measurement unit that measures an arbitrary parameter such as, for example, urine specific gravity, a pressure in the bladder, or a perfusion state around the urethra on the basis of information obtained by the sensor 38 disposed at a place not in contact with urine. By including the second measurement unit, it is possible to provide the measurement device 30 that measures a plurality of parameters in parallel.

The measurement system 10 does not have to include the temperature sensor 388 and the temperature measurement equipment 368. The measurement system 10 does not have to include the flow rate sensor 389 and the flow rate measurement equipment 369.

### Second Embodiment

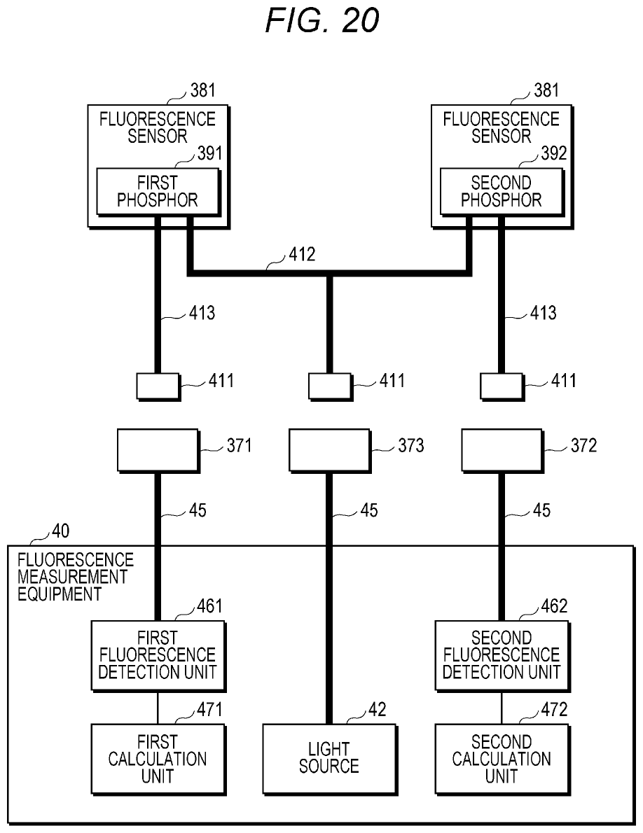

FIG. 20 is an explanatory view for explaining a configuration of the fluorescence measurement equipment 40 of the second embodiment. The fluorescence measurement equipment 40 of the present embodiment can detect an abnormality in measurement data of the urinary oxygen partial pressure. Description of parts common to the first embodiment will be omitted.

The fluorescence measurement equipment 40 according to the present embodiment includes one light source 42 and two fluorescence detection units 46 including a first fluorescence detection unit 461 and a second fluorescence detection unit 462. A first calculation unit 471 is connected to the first fluorescence detection unit 461. The first fluorescence detection unit 461 is connected to the first connector 371 through the light guide path 45. The second calculation unit 472 is connected to the second fluorescence detection unit 462. The second fluorescence detection unit 462 is connected to the second connector 372 through the light guide path 45. The light source 42 is connected to the third connector 373 via the light guide path 45.

Two fluorescence sensors 381 are connected to the fluorescence measurement equipment 40. One fluorescence sensor 381 includes a first phosphor 391. The other fluorescence sensor 381 includes a second phosphor 392. Both of the first phosphor 391 and the second phosphor 392 are phosphor 39 using the same fluorescent material that emits fluorescence according to the oxygen partial pressure in the urine.

The fluorescence sensor 381 having the first phosphor 391 is disposed in the vicinity of the side hole 151 and comes into contact with the urine in the bladder. The fluorescence sensor 381 is an example of a first sensor disposed in the bladder. The fluorescence sensor 381 having the second phosphor 392 can be disposed, for example, in the vicinity of the urination funnel 154 or inside the bag 171 and comes into contact with the urine led out of the patient's body. The fluorescence sensor 381 is an example of a second sensor disposed outside the body.

The first phosphor 391 is connected to the optical fiber connector 411 via the fiber for light reception 413. The second phosphor 392 is connected to the optical fiber connector 411 via the fiber for light reception 413. The first phosphor 391 and the second phosphor 392 are connected to the same optical fiber connector 411 via the bifurcated fiber for irradiation 412.

The excitation light emitted from the light source 42 irradiates the first phosphor 391 and the second phosphor 392 via the light guide path 45 and the fiber for irradiation 412. In a case where each of the first phosphor 391 and the second phosphor 392 comes into contact with urine, fluorescence corresponding to an oxygen partial pressure or an oxygen concentration in the urine is emitted.

The fluorescence emitted by the first phosphor 391 is incident on the first fluorescence detection unit 461 via the fiber for light reception 413 and the light guide path 45. The first fluorescence detection unit 461 converts the fluorescence into an electrical signal. The first calculation unit 471 analyzes the electrical signal and outputs the urinary oxygen partial pressure or the oxygen concentration to the bus in real time.

The fluorescence emitted by the second phosphor 392 is incident on the second fluorescence detection unit 462 via the fiber for light reception 413 and the light guide path 45. The second fluorescence detection unit 462 converts the fluorescence into an electrical signal. The second calculation unit 472 analyzes the electrical signal and outputs the urinary oxygen partial pressure or the oxygen concentration to the bus in real time.

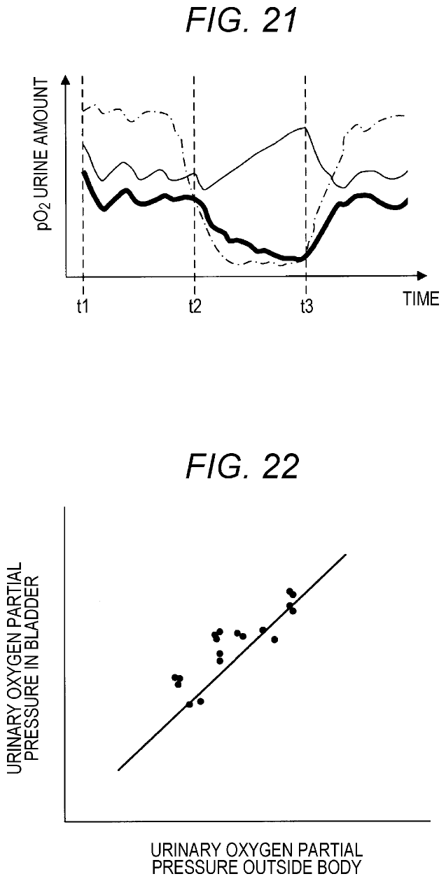

FIGS. 21 and 22 are explanatory views for explaining a method of detecting an abnormality in a measurement result. FIG. 21 is a graph schematically indicating temporal changes in a urinary oxygen partial pressure in the bladder, a urinary oxygen partial pressure outside the body, and a urine flow rate. FIG. 21 indicates time on a horizontal axis. FIG. 21 indicates the urinary oxygen partial pressure and the urine flow rate on a vertical axis. This is a schematic view, and thus, the unit of each axis is omitted.

A thick solid line indicates temporal change of the urinary oxygen partial pressure measured in the bladder. A thin solid line indicates temporal change of the urinary oxygen partial pressure measured outside the body. An alternate long and short dash line indicates temporal change of the urine flow rate. In a period from time t1 to time t2 and after time t3, trend of the urinary oxygen partial pressure in the bladder almost matches trend of the urinary oxygen partial pressure outside the body. However, in a period from time t2 to time t3, the urinary oxygen partial pressure tends to decrease, and the bladder external oxygen partial pressure tends to increase. During this period, the urine flow rate is relatively low.

For example, there is a case where oxygen is consumed by substances in urine, and the urinary oxygen partial pressure decreases. This phenomenon is likely to occur in retained urine. For example, in a case where urine is in an environment in which oxygen in the atmosphere is dissolved before measurement, the urinary oxygen partial pressure may increase. In any case, data to be originally measured cannot be measured. Thus, as illustrated in FIG. 21, in a case where trend of the measurement result differs depending on a place where the sensor 38 is disposed, reliability of the measurement data is relatively low.

FIG. 22 is a graph schematically indicating a relationship between the urinary oxygen partial pressure measured in the bladder and the urinary oxygen partial pressure measured outside the body. FIG. 22 indicates the urinary oxygen partial pressure in the bladder measured using the first phosphor 391 on a vertical axis. FIG. 22 indicates the urinary oxygen partial pressure outside the body measured using the second phosphor 392 on a horizontal axis. This is a schematic view, and thus, the unit of each axis is omitted.

Each black circle indicates measurement data measured substantially simultaneously. A thin solid line indicates a regression line calculated by, for example, a least squares method. In FIG. 22, trend of the urinary oxygen partial pressure in the bladder matches trend of the urinary oxygen partial pressure outside the body, and the slope of the regression line is positive. In a case where a graph similar to that in FIG. 21 is created using the data in the period from the time t2 to the time t3 in FIG. 22, the slope of the regression line becomes negative.

The slope of the regression line calculated using several measurement data from the latest one can be used as an index indicating reliability of the latest urinary oxygen partial pressure. The control unit 31 may display an index indicating the reliability together with the urinary oxygen partial pressures in the bladder and outside the body. The control unit 31 may display an index indicating the reliability together with one of the urinary oxygen partial pressures. In a case where only one is displayed, it can be desirable to display the urinary oxygen partial pressure in the bladder. The control unit 31 may change color or a size of a font for displaying the urinary oxygen partial pressure in the bladder according to the index. For example, in a case where the reliability is relatively low, the control unit 31 displays the urinary oxygen partial pressure in light color or a small font.

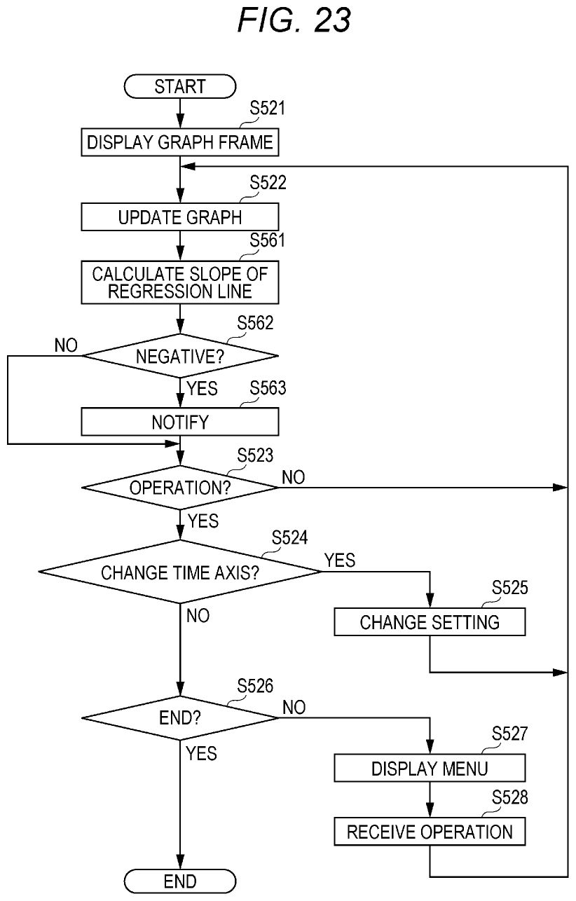

FIG. 23 is a flowchart for explaining flow of processing of a program of the second embodiment. The program of FIG. 23 is executed instead of the program described with reference to FIG. 6.

The control unit 31 displays a graph on the touch panel 35 (S521). The control unit 31 reads data from the measurement value DB 51 and updates the graph (S522). The control unit 31 calculates the slope of the regression line described with reference to FIG. 22 on the basis of the data corresponding to several times from the latest one (S561).

The control unit 31 determines whether or not the slope of the regression line is negative (S562). In a case where it is determined that the value is negative (YES in S562), the control unit 31 makes a notification that the reliability of the measurement value is relatively low (S563). The notification can be made, for example, by display on the touch panel 35 or sound output from the measurement device 30. The control unit 31 may transmit the notification to a nurse station, or the like, via a network such as HIS.

In a case where it is determined that the value is not negative (NO in S562), or after the processing of S563 is finished, the control unit 31 determines whether or not operation by the user has been received (S523). Subsequent processing is the same as the flow of the processing described with reference to FIG. 6, and thus, the description of the flow of the subsequent processing, for example, as described in FIG. 6 will be omitted.

In S562, the processing of the control unit 31 may proceed to S563 in a case where the slope of the regression line has changed. It is possible to provide the measurement system 10 that detects an abnormality of the measurement data at a relatively early stage.

According to the present embodiment, it is possible to provide the measurement system 10 that displays the reliability of the measurement data. The user can make an appropriate decision without being confused by data with relatively low reliability. In a case where the reliability is relatively low, the user may perform procedure necessary for increasing the reliability, such as correcting a position of the sensor 38.

According to the present embodiment, it is possible to provide the measurement system 10 that measures the urinary oxygen partial pressure at two sites using one light source 42. The parameter measured at each of the two sites is not limited to the urinary oxygen partial pressure. For example, any parameter may be measured at two sites.

Different fluorescent materials may be used for the first phosphor 391 and the second phosphor 392. A single light source 42 can be used to provide the measurement system 10 that acquires two types of measurement data. The two fluorescence sensors 381 may be disposed relatively close to each other or may be disposed away from each other.

In this case, the excitation light emitted from the light source 42 includes both a wavelength for exciting the first phosphor 391 and a wavelength for exciting the second phosphor 392. In a case where a wavelength of excitation light and a wavelength of the fluorescence are relatively close to each other, the excitation light can be separated from the fluorescence by appropriately using an optical filter.

### Third Embodiment

The present embodiment relates to the measurement system 10 that displays an index calculated on the basis of measured parameters. Description of parts common to the first embodiment will be omitted.

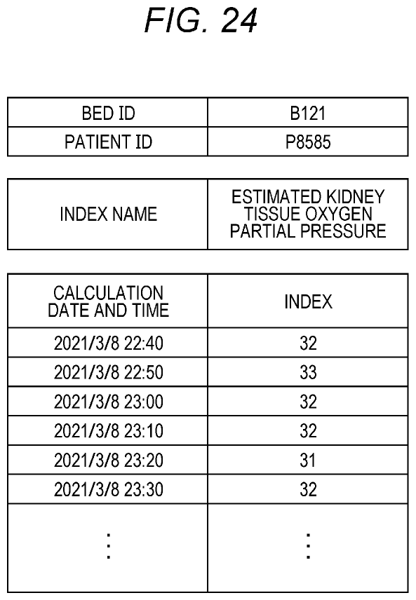

FIG. 24 is an explanatory view illustrating record layout of the index database (DB). The index DB is a database (DB) that records a bed ID, a patient ID, index name, calculation date and time, and an index in association with each other.

The index DB can include a bed ID field, a patient ID field, an index name field, a calculation date and time field, and an index field. A bed ID uniquely assigned to a bed is recorded in the bed ID field. In the patient ID field, a patient ID uniquely assigned to a patient is recorded.

In the index name field, index name is recorded. In FIG. 24, the index name is an “estimated kidney tissue oxygen partial pressure”. In the calculation date and time field, the date and time when the index is calculated is recorded. In the calculation date and time field, the measurement date and time of the most recently measured parameter among the plurality of parameters used to calculate the index may be recorded. In the index field, an index calculated on the basis of a plurality of parameters is recorded.

The “estimated kidney tissue oxygen partial pressure” indicated in FIG. 24 is an oxygen partial pressure in the kidney calculated on the basis of parameters such as the urinary oxygen partial pressure measured in the bladder, or the like. A formula for calculating the estimated kidney tissue oxygen partial pressure is determined on the basis of data measured in advance for a relatively large number of subjects or animals. The estimated kidney tissue oxygen partial pressure may be output by a machine-learned model so as to output the estimated kidney tissue oxygen partial pressure using inputs of attributes such as age and gender of the patient and the measured parameters. The “estimated kidney tissue oxygen partial pressure” is an example of an index calculated on the basis of the measured parameters.

The index may be a dissolved oxygen concentration dissolved in urine. A method for calculating the dissolved oxygen concentration on the basis of the urinary oxygen partial pressure is known, and thus, the description of the method for calculating the dissolved oxygen concentration on the basis of the urinary oxygen partial pressure is omitted. The control unit 31 may calculate and display an index defined by the user. The control unit 31 may calculate and display a plurality of indexes in parallel. The control unit 31 may change the index to be calculated according to the item for which the measurement data is obtained.

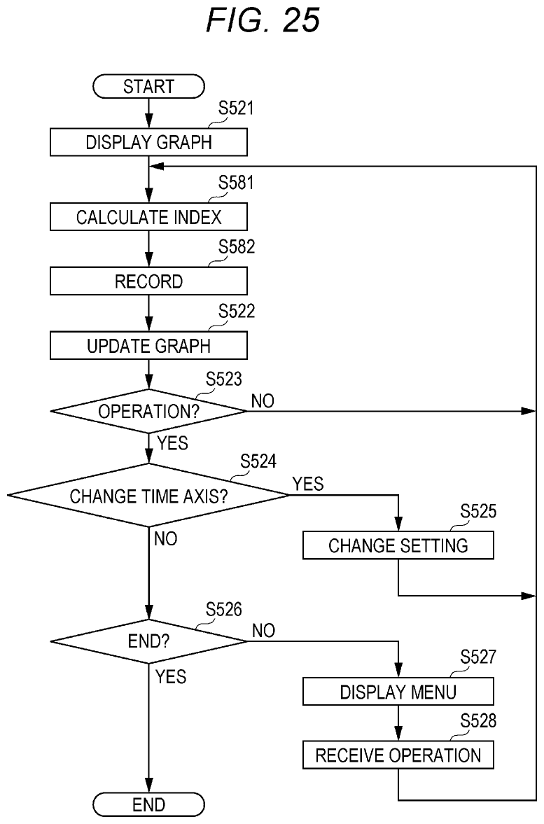

FIG. 25 is a flowchart for explaining flow of processing of the program of the third embodiment. The program of FIG. 25 is executed instead of the program described with reference to FIG. 6.

The control unit 31 displays a graph on the touch panel 35 (S521). The control unit 31 calculates an index on the basis of predetermined definition (S581). In a case where the number of pieces of data recorded in the measurement value DB 51 is insufficient and the index cannot be calculated, the control unit 31 waits until data of a necessary number of pieces is recorded in the measurement value DB 51.

The control unit 31 creates a new record in the index DB and records the calculated index and the calculation date and time (S582). Subsequent processing is the same as the flow of the processing described with reference to FIG. 6, and thus, the description of the subsequent processing will be omitted.

In S522, the control unit 31 may read data from the index DB and display or update a graph relating to the index.

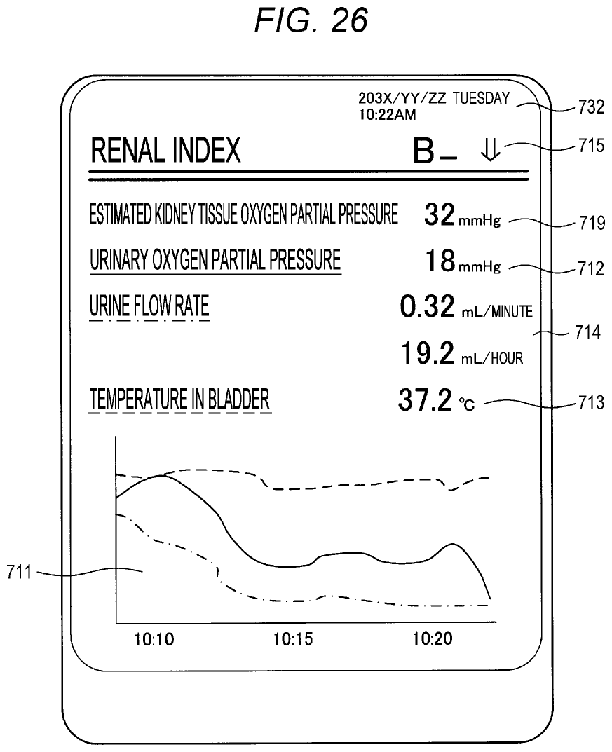

FIG. 26 is an explanatory view illustrating an example of a display screen according to the third embodiment. In FIG. 26, a renal index field 715 and an estimated kidney tissue oxygen partial pressure field 719 are added to the screen of the modification of the first embodiment described using FIG. 14.

The estimated kidney tissue oxygen partial pressure described above is displayed in the estimated kidney tissue oxygen partial pressure field 719. In the renal index field, an index related to a condition of the kidney determined on the basis of the parameter measured using each sensor 38 is displayed. After the index is calculated on the basis of a predetermined calculation formula, the control unit 31 displays a result of ranking into, for example, “A+”, “A”, “A−”, “B+”, “B”, “B−”, and the like, in the renal index field 715. A downward arrow at the right end of the renal index field 715 indicates that the index tends to decrease.

As indicated in the renal index field 715, by displaying the ranked indexes, it is possible to provide the measurement system 10 capable of intuitively confirming a comprehensive determination result using the plurality of parameters.

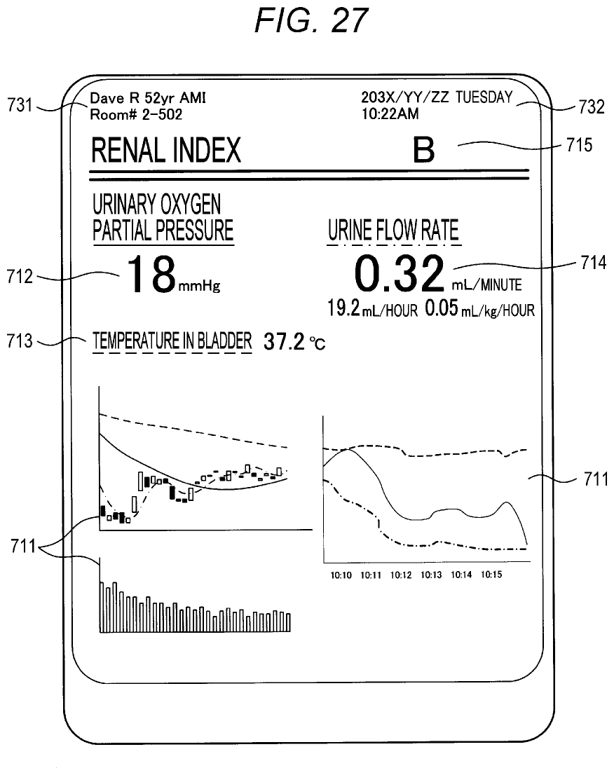

FIG. 27 is an explanatory view illustrating an example of the display screen according to the third embodiment. In FIG. 27, the renal index field 715 is added to the screen of the modification of the first embodiment described using FIG. 13. In the graph field 711 of FIG. 27, the candlestick chart described using FIG. 15 and a bar graph indicating an arbitrary index are added. In a case where the candlestick chart and a line graph are used, the user can relatively easily understand the trend of the index. In a case where a bar graph is used, the user can relatively easily understand a total amount per unit time. It is desirable that the user can appropriately change a format of the graph field 711 according to the purpose of use of the measurement system 10.

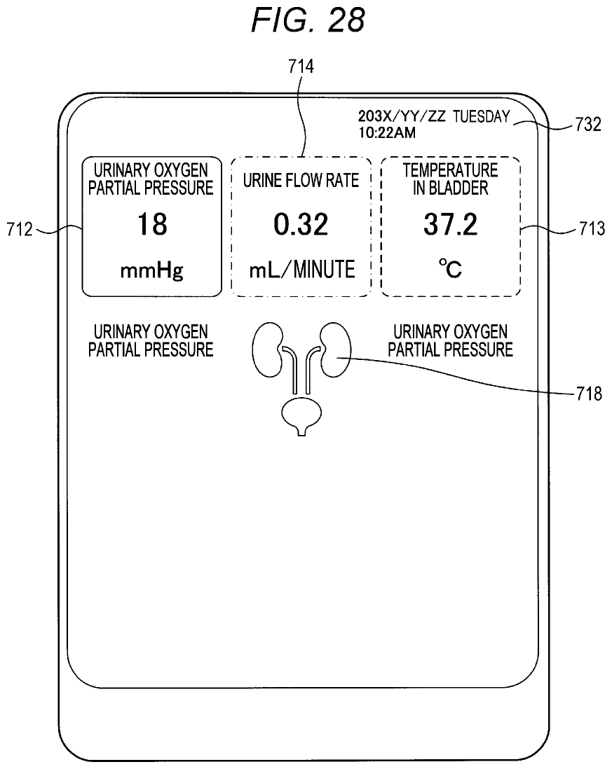

FIG. 28 is an explanatory view illustrating an example of the display screen according to the third embodiment. In FIG. 28, the oxygen partial pressure field 712, the urine flow rate field 714, and the renal index field 715 are displayed in a so-called tile format at the upper part of the screen. The kidney icon 718 is displayed at the center of the screen.

The control unit 31 determines a display form of the kidney icon 718 on the basis of an arbitrary index or parameter such as the renal index. For example, the control unit 31 displays the kidney icon 718 in blue in a case where the renal index is A rank, in black in a case where the renal index is B rank, and in red in a case where the renal index is C rank.

The index calculated by the control unit 31 may be a urine velocity per body weight or a mass velocity of oxygen flowing in the bladder indwelling catheter 15 per body weight. The index calculated by the control unit 31 may be a urine velocity per body surface area or a mass velocity of oxygen flowing in the bladder indwelling catheter 15 per body surface area. The body surface area of the patient is calculated, for example, on the basis of the height and weight of the patient. The control unit 31 implements a function of a velocity calculation unit that calculates these velocities.

According to the present embodiment, it is possible to provide the measurement system 10 that displays various indexes that support determination of the user.

### Fourth Embodiment

The present embodiment relates to the measurement system 10 that cooperates with various devices. Description of parts common to the first embodiment will be omitted.

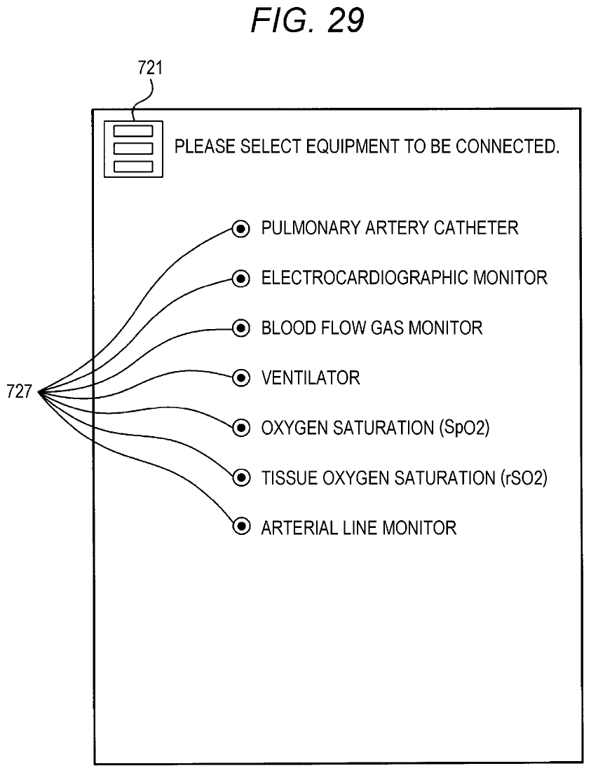

FIG. 29 is an explanatory view illustrating an example of a display screen according to the fourth embodiment. FIG. 29 is an example of a screen to be displayed by the control unit 31 in a case where the user gives an instruction to display a screen for selecting a device to cooperate. A list of devices connectable to the measurement device 30 via the HIS, or the like, is displayed. The connection equipment selection button 727 is displayed on the left side of the name of each device. The user can select equipment to be connected via the connection equipment selection button 727. In FIG. 29, all the displayed equipment is selected.

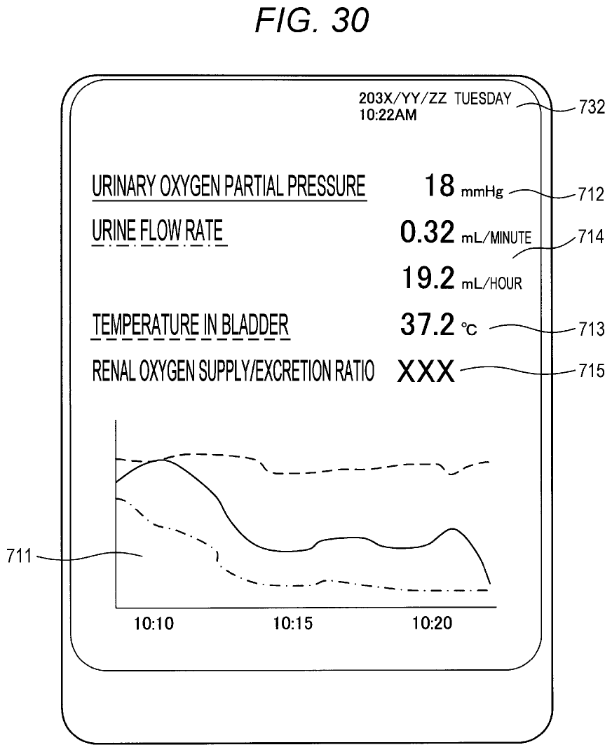

FIG. 30 is an explanatory view illustrating an example of the display screen according to the fourth embodiment. In FIG. 30, the renal index field 715 is added to the screen of the modification of the first embodiment described using FIG. 14. A renal oxygen supply/excretion ratio is displayed in the renal index field 715 of FIG. 30.

The renal oxygen supply/excretion ratio is calculated by the following formula.

Renal oxygen supply/excretion ratio=urinary oxygen mass velocity/arterial oxygen content×cardiac output

The urinary oxygen mass velocity can be calculated by a known calculation method on the basis of the urinary oxygen concentration and the urine flow rate. As described above, the urinary oxygen concentration can be calculated by a known calculation method on the basis of the urinary oxygen partial pressure. The control unit 31 acquires the arterial oxygen content and the cardiac output from the pulmonary artery catheter 194 or a blood gas monitoring device. In other words, the control unit 31 functions as a pulmonary artery catheter data acquisition unit that acquires data measured using the pulmonary artery catheter 194.

The renal oxygen supply/excretion ratio is dimensionless. By using milligrams/minute for the unit of the urinary oxygen mass velocity, milligrams/deciliters for the unit of the arterial oxygen content, and liters/minute for the unit of the cardiac output, it is possible to provide the measurement system 10 in which the user can rather easily understand a fluctuation state of the renal oxygen supply/excretion ratio.

The renal oxygen supply/excretion ratio of the patient during extracorporeal circulation is calculated by the following formula.

Renal oxygen supply/excretion ratio=urinary oxygen mass velocity/arterial oxygen content×pump flow rate

The control unit 31 acquires the pump flow rate from the extracorporeal circulation device 193. As the unit of the pump flow rate, liters/minute is used similarly to the unit of the cardiac output described above, so that it is possible to provide the measurement system 10 in which the user can rather easily understand a fluctuation state of the renal oxygen supply/excretion ratio.

The renal oxygen supply/excretion ratio is an example of an index for estimating an oxygen supply/demand state of the kidney. By calculating the renal oxygen supply/excretion ratio, the control unit 31 implements a function of an oxygen supply and demand estimation unit that estimates an oxygen supply and demand state of the kidney.

The control unit 31 may determine the oxygen supply and demand state of the kidney on the basis of the data measured using the pulmonary artery catheter 194 and the data measured using the sensor 38. The control unit 31 implements a function of an oxygen supply and demand unit that determines an oxygen supply and demand state of the kidney.

The control unit 31 may display a renal oxygen excretion rate in the renal index field 715. The renal oxygen excretion rate indicates an amount of oxygen dissolved in urine and excreted from the kidney and is calculated by the following formula.

Renal oxygen excretion rate=urinary oxygen concentration×urine flow rate

By using milligrams/milliliters for the unit of the urinary oxygen concentration and milliliters/minute for the unit of the urine flow rate, it is possible to provide the measurement system 10 in which the user can rather easily grasp (or understand) a fluctuation state of the renal oxygen excretion rate.

The control unit 31 may display a central renal oxygen partial pressure ratio in the renal index field 715. The renal oxygen partial pressure ratio is calculated by the following formula.

Central renal oxygen partial pressure ratio=urinary oxygen concentration/arterial oxygen content

By using milligrams/liter for the unit of the urinary oxygen concentration and milligrams/deciliters for the unit of the arterial oxygen content, it is possible to provide the measurement system 10 in which the user can rather easily grasp (or understand) a fluctuation state of the central renal oxygen partial pressure ratio.

The control unit 31 may display the renal oxygen supply/excretion ratio during extracorporeal circulation in the renal index field 715. The renal oxygen supply/excretion ratio during extracorporeal circulation indicates a ratio between an amount of oxygen sent from the extracorporeal circulation device 193 during the extracorporeal circulation and the above-described renal oxygen excretion rate. The amount of oxygen sent from the extracorporeal circulation device 193 is acquired from the extracorporeal circulation device 193.

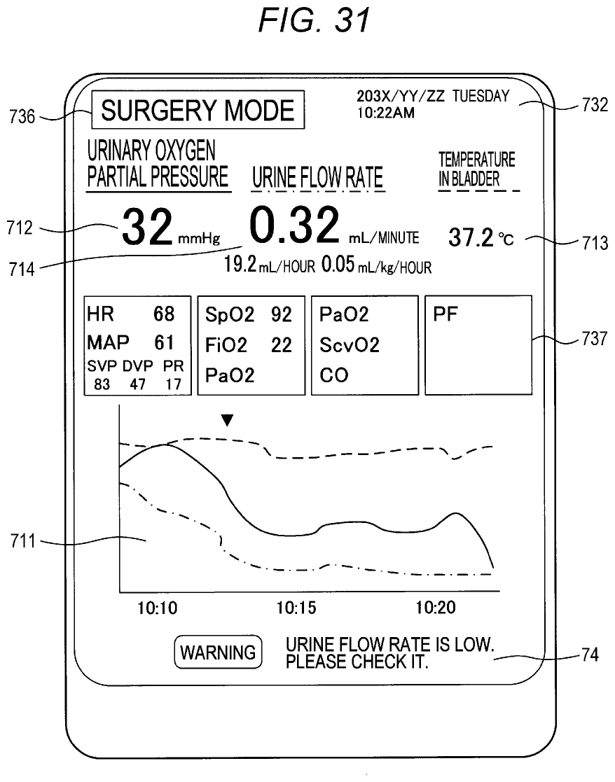

FIG. 31 is an explanatory view illustrating an example of the display screen according to the fourth embodiment. In FIG. 31, a mode field 736 and an other device field 737 are added to the screen according to the modification of the first embodiment described with reference to FIG. 13.

In the mode field 736, name of a mode determined for each layout of the screen or for each setting of the measurement device 30 is displayed. In FIG. 31, a “surgical mode” suitable for use during surgery is displayed.

In the other device field 737, various kinds of data acquired from devices such as the vital measurement device 191 are displayed together with abbreviations. The data displayed in the other device field 737 can include, for example, a blood storage amount, an infusion amount, a bleeding amount, a blood transfusion amount, a perfusion flow rate, or a perfusion pressure during extracorporeal circulation operation acquired from the extracorporeal circulation device 193. The control unit 31 implements a function of an extracorporeal circulation data acquisition unit that acquires these data from the extracorporeal circulation device 193. The extracorporeal circulation data acquisition unit is an example of an external data acquisition unit.

The data displayed in the other device field 737 can include, for example, an oxygen partial pressure of the circulating blood, a carbon dioxide partial pressure, a potassium concentration, a sodium concentration, a hydrogen ion concentration index, an oxygen supply amount, oxygen saturation, a hematocrit value, a hemoglobin amount, an oxygen consumption amount, a plasma bicarbonate ion concentration, or base excess. The control unit 31 implements a function of a circulating blood data acquisition unit that acquires these data from a device such as the vital measurement device 191. The circulating blood data acquisition unit is an example of an external data acquisition unit.

According to the screen example illustrated in FIG. 31, the user can save time and effort to check the screens of the devices arranged in various places.

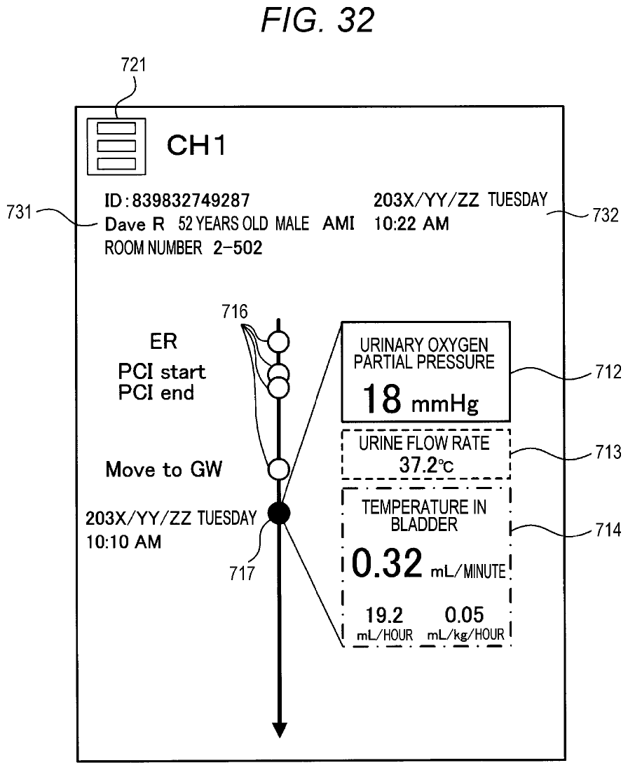

FIG. 32 is an explanatory view illustrating an example of the display screen according to the fourth embodiment. In FIG. 32, the oxygen partial pressure field 712, the temperature field 713, and the urine flow rate field 714 are displayed together with the outline of a progress status of the surgery.

A downward arrow in the center of the screen illustrated in FIG. 32 indicates passage of time. An event index 716 indicated by a white circle indicates that a process serving as a point of the surgery has been executed. An abbreviation or an icon indicating content of an event is displayed on the left side of each event index 716.

The control unit 31 displays the event index 716 on the basis of information acquired from a surgery management system, or the like, that records progress of surgery. The control unit 31 may display the event index 716 on the basis of an instruction given by the user for each generated event.

A latest measurement index 717 indicated by a black circle indicates a timing at which the latest measurement by the measurement device 30 is performed. The date and time when the latest measurement is performed is displayed on the left side of the latest measurement index 717. By sliding the black circle on the time axis, the user can check data at any time and grasp (or understand) the state of the patient.

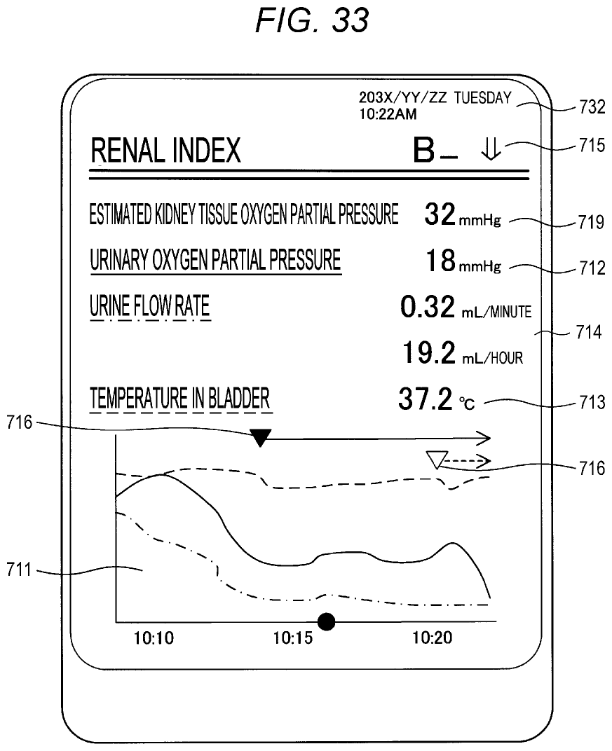

FIG. 33 is an explanatory view illustrating an example of the display screen according to the fourth embodiment. In FIG. 33, the event index 716 is displayed in a superimposed manner in the graph field 711 of the screen described with reference to FIG. 26. The event index 716 indicates a type of the event that has occurred by a shape such as a black inverted triangle, a white inverted triangle, and a black circle.

The arrows extending rightward from the event indexes 716 indicated by the black inverted triangle and the white inverted triangle indicate that the events are continuing. The event index 716 indicated by the black circle ends in a short period, and thus, the arrow does not extend. Events include blood transfusion, medication, interventions, a timing of data extraction, initiation and termination of extracorporeal circulation during the surgery, insertion and removal of the pulmonary artery catheter 194, use of the ventilator 192, and the like. The user can confirm what kind of event has occurred by touching the event index 716. For example, in a case where the event is medication, simple information such as prescription of the medication is displayed in a pop-up.

With the screens illustrated in FIGS. 32 and 33, it is possible to provide the measurement system 10 that allows the user to confirm the parameters measured by the measurement device 30 together with the flow of the entire surgery.

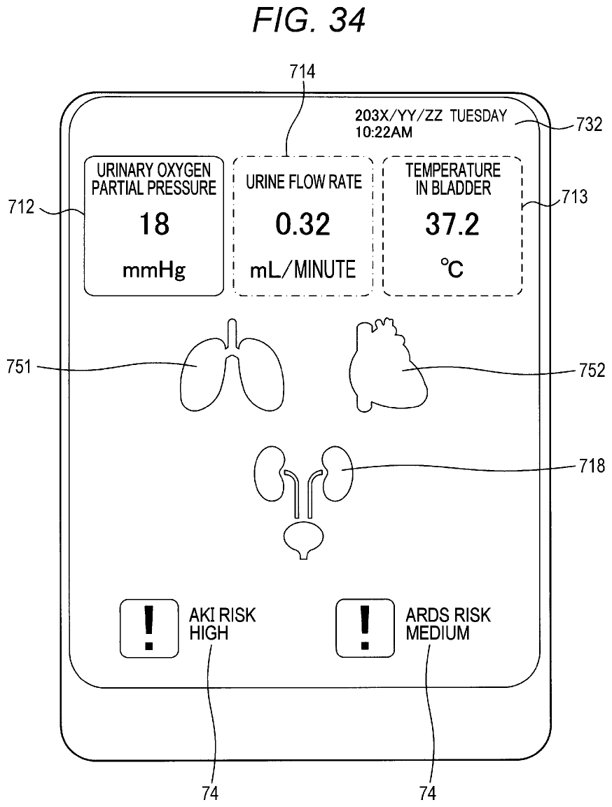

FIG. 34 is an explanatory view illustrating an example of the display screen according to the fourth embodiment. In FIG. 34, a lung icon 751, a heart icon 752, and the kidney icon 718 are displayed on the screen described with reference to FIG. 28. In the lower part of the screen, the notification field 74 indicating that a risk of occurrence of acute kidney injury (AKI) is relatively high, and the notification field 74 indicating that a risk of occurrence of “acute respiratory distress syndrome (ARDS)” is medium is displayed.

The control unit 31 highlights an icon indicating an organ deeply related to the risk displayed in the notification field 74 by coloring, blinking, or the like.

The control unit 31 determines the AKI risk and the ARDS risk on the basis of, for example, an index calculated on the basis of the parameters measured using the sensor 38 and data acquired from other devices and displays the notification field 74 in a case where the risk is relatively high. The control unit 31 may display information acquired from other devices in the notification field 74, or the like.

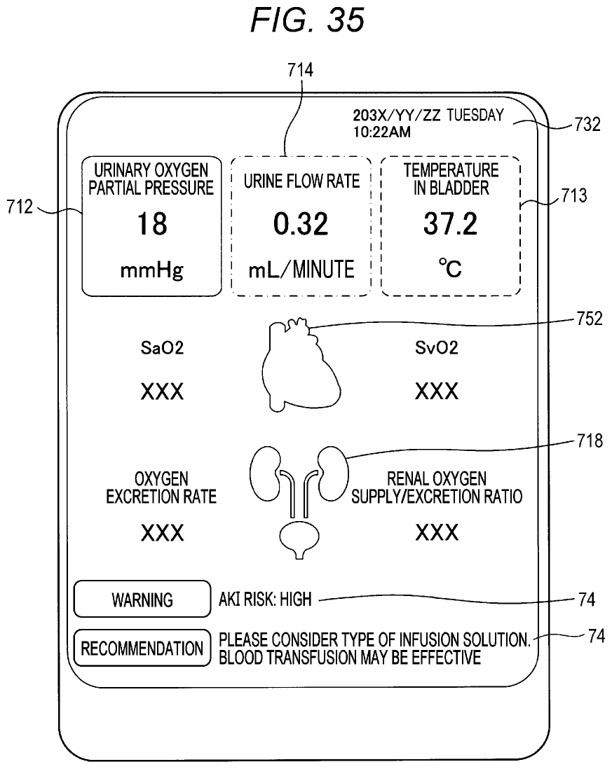

FIG. 35 is an explanatory view illustrating an example of the display screen according to the fourth embodiment. In FIG. 35, the heart icon 752 and the kidney icon 718 are displayed on the screen described with reference to FIG. 28. Values of SaO2 (arterial oxygen saturation) and SvO2 (mixed venous oxygen saturation) are displayed on the left and right of the heart icon 752. The oxygen excretion rate described in FIG. 30 and the renal oxygen supply/excretion ratio described in FIG. 26 are displayed on the left and right of the kidney icon 718.

In the lower part of the screen, the notification field 74 indicating that a risk of the occurrence of AKI is high and a message like “please consider a type of infusion solution. Blood transfusion may be effective”, and the notification field 74 indicating that infusion or blood transfusion is recommended are displayed.

The control unit 31 determines the AKI risk and the recommended treatment on the basis of, for example, the index calculated on the basis of the parameters measured using the sensor 38 and data acquired from other devices and displays the notification field 74. The control unit 31 may display information acquired from other devices in the notification field 74, or the like.

For example, the control unit 31 may acquire administration data of a diuretic from the drug administration device 196 and determine change in the urine flow rate associated with the administration of the diuretic, change in the urinary oxygen partial pressure, or the like. The control unit 31 may estimate a state of the renal function on the basis of the change in the urine flow rate, the change in the urinary oxygen partial pressure, or the like. In this case, the control unit 31 implements a function of a diuretic data acquisition unit that acquires administration data of the diuretic and a function of a renal function estimation unit that estimates the renal function on the basis of the change in the urine flow rate or the change in the urinary oxygen partial pressure associated with the administration of the diuretic. The diuretic data acquisition unit is an example of a drug administration data acquisition unit.

The control unit 31 may have a function of supporting various examinations such as an examination regarding diuretic responsiveness or an examination regarding infusion responsiveness. The examination regarding diuretic responsiveness will be described as an example. The user administers a predetermined amount of a diuretic such as furosemide to the patient.

The control unit 31 acquires time when the diuretic is administered from the drug administration device 196. The control unit 31 displays, on the display unit 351, the urine flow rate and the urinary oxygen partial pressure within a predetermined period from the time when the diuretic is administered in a mode (or manner) that can be compared with the data before the diuretic is administered. The control unit 31 may display the urinary oxygen partial pressure on the display unit 351 together with the urine flow rate.

The user can confirm change in the urine flow rate due to the diuretic, for example, the patient's diuretic responsiveness. For example, in a case where the amount of the urine after administration of the diuretic is equal to or less than a reference amount, the user suspects that renal dysfunction has progressed. The control unit 31 may support determination of the user by displaying the reference amount on the display unit 351 together with the urine flow rate of the patient. The user can confirm a load exerted by the diuretic on the renal parenchyma on the basis of the change in the urinary oxygen partial pressure. As described above, it is possible to provide the measurement device 30 that supports the examination regarding the diuretic responsiveness.

The control unit 31 may display a diuretic effect assessment index calculated by the following formula.

Diuretic effect assessment index=urine flow rate×elapsed time/diuretic dose

In the above formula, the elapsed time indicates the elapsed time after administration of the diuretic. By using milliliters/minute as the unit of the urine flow rate, minutes as the unit of the elapsed time, and milligrams as the unit of the diuretic dose, it is possible to provide the measurement system 10 in which the user can rather easily grasp (or understand) a fluctuation state of the renal oxygen supply/excretion ratio.

The control unit 31 may display a second diuretic effect assessment index calculated by the following formula.

Second diuretic effect assessment index=urinary oxygen mass velocity/diuretic effect assessment index

The second diuretic effect assessment index is dimensionless. By using milligram/minute as a unit of the urinary oxygen mass velocity and milliliter/milligram as a unit of the diuretic effect assessment index, it is possible to provide the measurement system 10 in which the user can rather easily grasp (or understand) a fluctuation state of the renal oxygen supply/excretion ratio.

The control unit 31 may display a sodium excretion rate calculated by the following formula.

Sodium excretion rate=urinary sodium concentration×urine flow rate

By using milligrams/milliliters for the unit of the urinary sodium concentration and milliliters/minute for the unit of the urine flow rate, it is possible to provide the measurement system 10 in which the user can rather easily grasp (or understand) a fluctuation state of the sodium excretion rate.

The control unit 31 may determine body fluid balance of the patient on the basis of the parameters measured using the sensor 38 and data acquired from other devices. The control unit 31 implements a function of the body fluid balance determination unit.

The control unit 31 may output data in which a risk of deterioration in a renal function is predicted in an arbitrary period such as within seven days after surgery or within 90 days after surgery on the basis of the parameters measured using the sensor 38 and data acquired from other devices. In this case, the control unit 31 implements a function of a renal function deterioration risk prediction unit.

The control unit 31 may output a predicted value regarding a future rising level of the serum creatinine value on the basis of the parameters measured using the sensor 38 and data acquired from other devices. In this case, the control unit 31 implements a function of a serum creatinine prediction unit.

The control unit 31 may display a therapeutic guideline related to the patient on the basis of the parameters measured using the sensor 38, data acquired from other devices, guidelines for each disease, and the like.

The control unit 31 may calculate and display a recommended value of an inhaled oxygen concentration on the basis of the parameters measured using the sensor 38 and the inhaled oxygen concentration data acquired from the ventilator 192. In this case, the control unit 31 implements a function of an inhaled oxygen concentration data acquisition unit.

### Fifth Embodiment

The present embodiment relates to the fluorescence measurement equipment 40 that receives fluorescence emitted from two kinds of phosphors 39 through one optical fiber 41. Description of parts common to the third embodiment will be omitted.

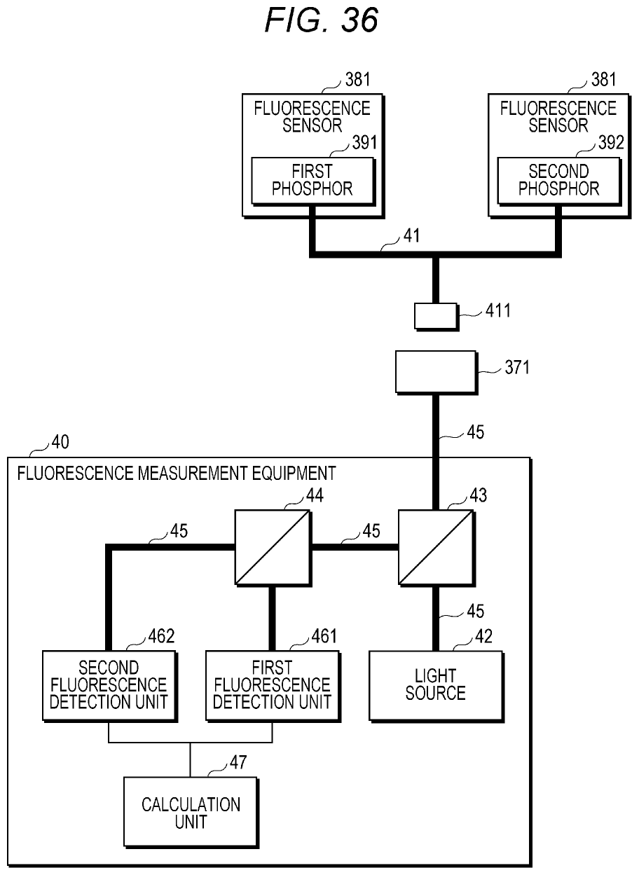

FIG. 36 is an explanatory view for explaining a configuration of the fluorescence measurement equipment 40 of the fifth embodiment. The fluorescence measurement equipment 40 according to the present embodiment includes one light source 42, the beam splitter 43, the second beam splitter 44, and two fluorescence detection units 46 including the first fluorescence detection unit 461 and the second fluorescence detection unit 462. The first fluorescence detection unit 461 and the second fluorescence detection unit 462 are connected to the identical calculation unit 47.

The light source 42 is connected to the beam splitter 43 via the light guide path 45. The beam splitter 43 is connected to the first connector 371 via the light guide path 45. The second beam splitter 44 is connected to the beam splitter 43 via the light guide path 45.

The first fluorescence detection unit 461 and the second fluorescence detection unit 462 are connected to the second beam splitter 44 via the light guide path 45. The second beam splitter 44 is a dichroic beam splitter that optically separates incident light on the basis of a wavelength.

Two fluorescence sensors 381 are connected to the fluorescence measurement equipment 40. One fluorescence sensor 381 includes a first phosphor 391. The other fluorescence sensor 381 includes a second phosphor 392. The first phosphor 391 and the second phosphor 392 emit fluorescence having different wavelengths in response to different components, or the like, in urine.

The two fluorescence sensors 381 may be disposed close to each other or may be disposed away from each other. One fluorescence sensor 381 may include the first phosphor 391 and the second phosphor 392.

The light source 42 emits excitation light that excites both the first phosphor 391 and the second phosphor 392. The excitation light emitted from the light source 42 irradiates the first phosphor 391 and the second phosphor 392 via the light guide path 45, the beam splitter 43, and the optical fiber 41. The first phosphor 391 and the second phosphor 392 each emit fluorescence in a case where the first phosphor 391 and the second phosphor 392 come into contact with urine.

The fluorescence is incident on the beam splitter 43 via the optical fiber 41 and the light guide path 45. By the beam splitter 43, the fluorescence is incident on the light guide path 45 connected to the second beam splitter 44. The second beam splitter 44 separates the fluorescence into the fluorescence emitted by the first phosphor 391 and other light. The fluorescence emitted from the first phosphor 391 is incident on the first fluorescence detection unit 461, and the other light is incident on the second fluorescence detection unit 462. The second beam splitter 44 functions as a spectroscopic unit that spectrally disperses the fluorescence acquired from the plurality of fluorescence sensors 381.

The first fluorescence detection unit 461 and the second fluorescence detection unit 462 convert the incident light into an electrical signal. The calculation unit 47 analyzes the electrical signal to calculate a measurement item corresponding to each fluorescence sensor 381. The calculation unit 47 functions as an analysis unit that analyzes each of the dispersed fluorescence on the basis of a predetermined algorithm.

### Sixth Embodiment

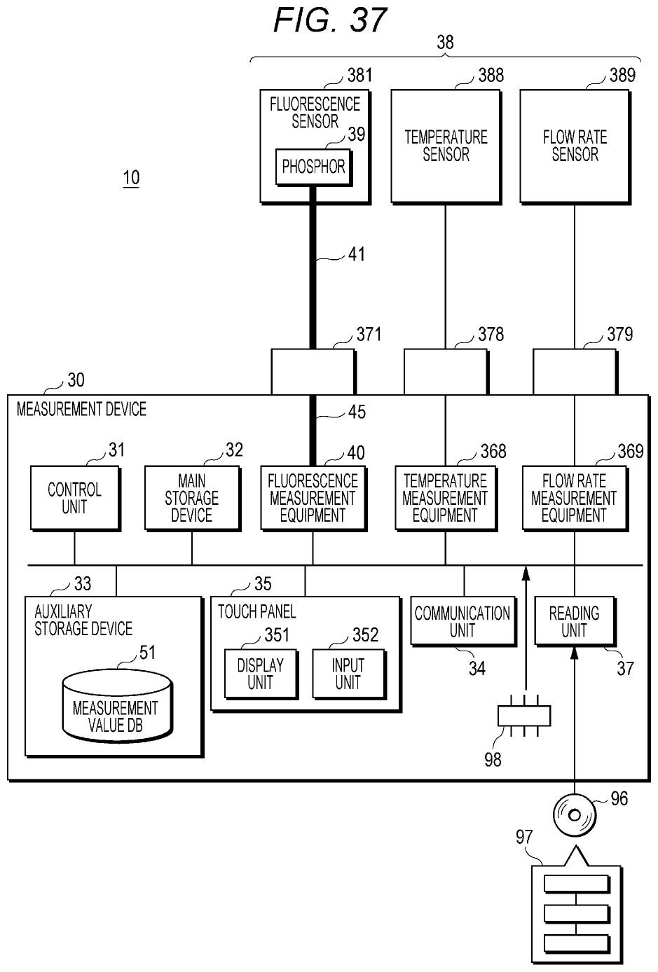

The present embodiment relates to a mode for implementing the measurement device 30 of the present embodiment by operating the program 97 recorded in a portable recording medium 96. FIG. 37 is an explanatory view for explaining a configuration of the measurement device 30 of the sixth embodiment.

The measurement device 30 of the present embodiment includes a reading unit 37. The program 97 is recorded in the portable recording medium 96. The control unit 31 reads the program 97 via the reading unit 37 and stores the program in the auxiliary storage device 33. In addition, the control unit 31 may read the program 97 stored in a semiconductor memory 98 such as a flash memory mounted in the measurement device 30. Furthermore, the control unit 31 may download the program 97 from another server computer connected via the communication unit 34 and a network and store the program in the auxiliary storage device 33.

The program 97 is installed as a control program of the measurement device 30, loaded into the main storage device 32, and executed.

### Seventh Embodiment

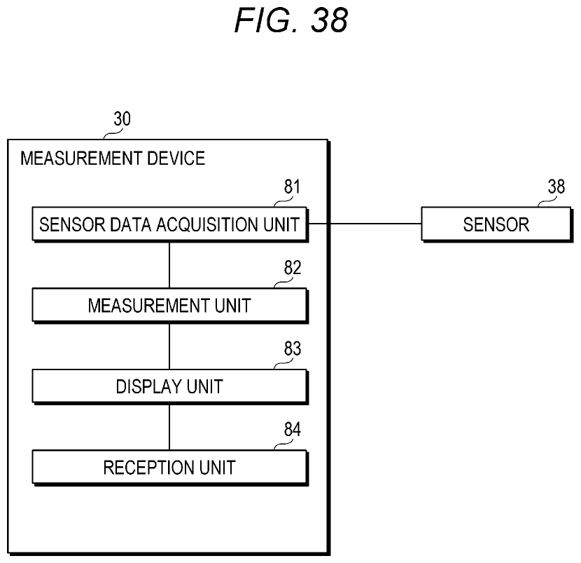

FIG. 38 is a functional block diagram of the measurement system 10 of a seventh embodiment. The measurement system 10 includes the sensor 38 and the measurement device 30. The sensor 38 is disposed so as to be able to come into contact with urine to be conducted by the catheter 15.

The measurement device 30 includes a sensor data acquisition unit 81, a measurement unit 82, a display unit 83, and a reception unit 84. The sensor data acquisition unit 81 acquires sensor data from the sensor 38. The measurement unit 82 sequentially measures parameters related to urine on the basis of the sensor data acquired by the sensor data acquisition unit 81. The display unit 83 displays the parameters in a graph using a time axis. The reception unit 84 receives a change instruction related to the time axis.

The technical features (components) described in the embodiments can be combined with each other, and new technical features can be formed by the combination.

The detailed description above describes embodiments of a measurement device, a measurement system, an information processing method, and a program. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A measurement device comprising:
a measurement unit configured to sequentially measure parameters related to urine on a basis of information obtained by a sensor disposed so as to be contactable with urine to be conducted by a catheter;
a display unit configured to display the parameters as a graph using a time axis; and
a reception unit configured to receive a change instruction related to the time axis.

2. The measurement device according to claim 1,
wherein the parameters include a urinary oxygen partial pressure; and
the measurement device comprises a correction unit configured to correct the urinary oxygen partial pressure measured by the measurement unit by at least one of an atmospheric pressure, a pressure in the bladder, a temperature, or chloride concentration in the urine.

3. The measurement device according to claim 1, further comprising:
a second measurement unit configured to measure at least one of urine specific gravity, a pressure in a bladder, a blood flow velocity around a urethra, or a urine flow rate on a basis of information obtained by a sensor disposed at a place not in contact with urine to be conducted by the catheter.

4. The measurement device according to claim 1, further comprising:
a velocity calculation unit configured to calculate a velocity of urine per body weight, a velocity of urine per body surface area, a mass velocity of oxygen flowing in the catheter per body weight, or a mass velocity of oxygen flowing in the catheter per body surface area.

5. The measurement device according to claim 1, wherein the sensor includes a first sensor disposed in the bladder and a second sensor disposed outside a body.

6. The measurement device according to claim 1, wherein the sensor includes two or more oxygen sensors and two or more temperature sensors.

7. The measurement device according to claim 1, further comprising:
an external data acquisition unit configured to acquire data measured using a ventilator, an extracorporeal circulation device, a pulmonary artery catheter, or a left ventricular catheter; and
wherein the display unit is configured to display the data acquired by the external data acquisition unit together with the graph indicating the parameters.

8. The measurement device according to claim 7, further comprising:
a patient information acquisition unit configured to acquire at least one of age, gender, body weight, a serum creatinine value, or a urea nitrogen amount; and
a serum creatinine prediction unit configured to predict a future rising level of the serum creatinine value on a basis of the data acquired by the patient information acquisition unit and the data acquired by the external data acquisition unit.

9. The measurement device according to claim 7, wherein in a case where the reception unit receives the change instruction related to the time axis, the external data acquisition unit is configured to acquire data corresponding to a range of the time axis changed on the basis of the change instruction.

10. The measurement device according to claim 1, further comprising:
an extracorporeal circulation data acquisition unit configured to acquire one or more of a blood storage amount, an infusion amount, a bleeding amount, a blood transfusion amount, a perfusion flow rate, or a perfusion pressure during extracorporeal circulation execution;
a circulating blood data acquisition unit configured to acquire one or more of an oxygen partial pressure, a carbon dioxide partial pressure, a potassium concentration, a sodium concentration, a hydrogen ion concentration index, an oxygen supply amount, an oxygen saturation, a hematocrit value, a hemoglobin amount, an oxygen consumption amount, a plasma bicarbonate ion concentration, or a base excess of a circulating blood; and
an oxygen supply and demand estimation unit configured to estimate an oxygen supply and demand state of a kidney on a basis of the data acquired by the extracorporeal circulation data acquisition unit, the data acquired by the circulating blood data acquisition unit, and the parameters.

11. The measurement device according to claim 10, wherein in a case where the reception unit receives the change instruction related to the time axis, the extracorporeal circulation data acquisition unit and the circulating blood data acquisition unit acquire data corresponding to a range of the time axis changed on a basis of the change instruction.

12. The measurement device according to claim 1, further comprising:
a pulmonary artery catheter data acquisition unit configured to acquire one or more of an arterial oxygen content, a cardiac output, or an arterial oxygen saturation measured using a pulmonary artery catheter; and
an oxygen supply and demand unit configured to determine an oxygen supply and demand state of the kidney on a basis of the data acquired by the pulmonary artery catheter data acquisition unit and the parameters.

13. The measurement device according to claim 12, wherein in a case where the reception unit receives the change instruction related to the time axis, the pulmonary artery catheter data acquisition unit acquires data corresponding to a range of the time axis changed on a basis of the change instruction.

14. The measurement device according to claim 1, further comprising:
a drug administration data acquisition unit configured to acquire data from a drug administration device; and
a body fluid balance determination unit configured to determine body fluid balance on a basis of the data acquired by the drug administration data acquisition unit and the parameters.

15. The measurement device according to claim 14,
wherein the parameters include a urine flow rate or a urinary oxygen partial pressure;
the drug administration data acquisition unit includes a diuretic data acquisition unit configured to acquire administration data of a diuretic; and
the measurement device comprises a renal function estimation unit configured to estimate a renal function on a basis of change in the urine flow rate associated with administration of a diuretic or change in the urinary oxygen partial pressure associated with administration of a diuretic, the change being determined on a basis of the diuretic administration data acquired by the diuretic data acquisition unit.

16. The measurement device according to claim 14, wherein in a case where the reception unit receives the change instruction related to the time axis, the drug administration data acquisition unit acquires data corresponding to a range of the time axis changed on a basis of the change instruction.

17. The measurement device according to claim 1, further comprising:
an inhaled oxygen concentration data acquisition unit configured to acquire inhaled oxygen concentration data;
wherein the measurement device calculates a recommended value of the inhaled oxygen concentration on a basis of the inhaled oxygen concentration data acquired by the inhaled oxygen concentration data acquisition unit and the parameters; and
wherein in a case where the reception unit receives the change instruction related to the time axis, the inhaled oxygen concentration data acquisition unit acquires data corresponding to a range of the time axis changed on a basis of the change instruction.

18. The measurement device according to claim 1, further comprising:
a sensor eigenvalue acquisition unit that acquires a sensor eigenvalue defined uniquely for the sensor; and
a calibration unit that calibrates the sensor on a basis of the sensor eigenvalue.

19. A measurement system comprising:
a sensor, the sensor being disposed so as to be contactable with urine to be conducted by a catheter; and
a measurement device, the measurement device including:
a sensor data acquisition unit configured to acquire sensor data from the sensor; and
a measurement unit configured to sequentially measure parameters related to urine on a basis of the sensor data acquired by the sensor data acquisition unit;
a display unit configured to display the parameters as a graph using a time axis; and
a reception unit configured to receive a change instruction related to the time axis.

20. An information processing method comprising:
acquiring information from a sensor contactable with urine to be conducted by a catheter;
sequentially measuring parameters related to urine using the acquired information; and
displaying the parameters as a graph using a time axis; and
receiving a change instruction related to the time axis.
